SEQUENCE LISTING (7 sequences)

SEQ ID NO: 1 - protein, 13 aa, Artificial Sequence
  SEQ ID NO 1 is a commercially available substrate available from Upstate Cell Signaling Solutions, Inc. as product number 12-516
KKSRGDYMTMQIG

SEQ ID NO: 2 - protein, 5 aa, Artificial Sequence
  SEQ ID NO 2 is a repeating unit in poly([Glu]4Tyr), and includes 4 Glu residues and 1 Tyr residue in random order. Thus, each of residues Xaa at positions 1-5 is Glu or Tyr, provided that only one is Tyr and the remaining four are Glu.
XXXXX

SEQ ID NO: 3 - protein, 17 aa, Artificial Sequence
  SEQ ID NO3 is operable as a biotinylation recognition sequence that operates in a fusion protein to covalently bind biotin. SEQ ID NO 3 is a consensus sequence derived from proteins that are substrates for E. coli biotin ligase (BirA).
MSGLNDIFEAQKIEWHE

SEQ ID NO: 4 - protein, 25 aa, Artificial Sequence
  Synthetic peptide
MIYYHHHHHHDYGIPTTENLYFQAL

SEQ ID NO: 5 - protein, 11 aa, Artificial Sequence
  Synthetic Peptide
GRPRTSSFAEG

SEQ ID NO: 6 - protein, 14 aa, Artificial Sequence
  Synthetic Peptide
ARKRERTYSFGHHA

SEQ ID NO: 7 - protein, 38 aa, Artificial Sequence
  Synthetic peptide
PKTFCGTPEYLAPEVRREPRILSEEEQEMFRDFDYIAD

(19) United States
(12) Patent Application Publication
(10) Pub. No.: US 2012/0244566 A1
(43) Pub. Date: Sep. 27, 2012

(54) Methods and materials for in vitro analysis and/or use of membrane-associated proteins, portions thereof or variants thereof

(76) Inventors: Robert M. Weis, Amherst, MA (US); Anthony L. Shrout, Easthampton, MA (US); Edward A. Esposlto, Sunderland, MA (US)

(21) Appl. No.: 13/385,897

(22) Filed: Mar. 12, 2012

Related U.S. Application Data
(63) Continuation-in-part of application No. 11/823,728, filed on Jun. 28, 2007.
(60) Provisional application No. 61/486,063, filed on May 13, 2011.

(51) Int. Cl. C12Q 1/48

(52) U.S. Cl. 435/15

(57) ABSTRACT

Methods and materials use template-directed assembly of polypeptides and optionally additional reagents to analyze the functionality of membrane-associated proteins, such as, for example, portions of transmembrane proteins, membrane-associated proteins (including receptor tyrosine kinases, and non-receptor tyrosine and serine-threonine kinases), and other proteins that bind to transmembrane proteins and membrane-associated proteins, and to analyze the effect of test compounds or mutations on the functionality of same. The methods and materials of the present application provide a more native-like environment for analyzing the functionality of membrane-associated proteins, and thus provide effective tools for studies involving the detection of the level of enzyme activity of such proteins in an environment that closely resembles the native environment in the cell, and for novel manufacturing processes.

This application is a continuation-in-part of and claims priority benefit from application Ser. No. 11/823,728 filed on Jun. 28, 2007, and provisional application Ser. No. 61/486,063 filed on May 13, 2011, each of which is incorporated herein by reference in its entirety.

### BACKGROUND OF THE INVENTION

The present application relates to the field of biotechnology, and in particular, to fields involving the study and use of membrane-associated proteins.

All living organisms are composed of cells, from single celled organisms such as bacteria, to the complex cellular architecture of humans. The cells include multifaceted, chemically driven systems, such as, for example, communication networks that control a cell's response to external stimulus. Signal transduction pathways involve protein ‘teams’ that work in concert to execute desired pathway instructions, such as, for example, gene regulation, cell growth, movement, and hormone release.

Cell membranes are bilayers of lipid molecules that define the boundary between, and serve as selective barriers between, the inside and outside of all cells and between the inside and outside of cellular compartments (organelles). Similar membranes also define the boundary between the inside and outside of some viruses. A wide variety of proteins are embedded in or on, or associated with, the cell membrane, thereby creating a highly specialized environment. It is widely accepted that the membrane environment, including the proteins and assemblies of proteins that naturally occur in and on the membrane, is essential for normal biological function. For example, a significant portion of these membrane proteins are responsible for the process of transmembrane signaling, which conveys information across the membrane, frequently, although not exclusively, from the outside of the cell to the inside. The membrane can be likened to a two-dimensional fluid sheet, which serves as the natural template for the assembly of signal transduction elements. The association of these proteins with the membrane in essence restricts their motion to two dimensions rather than three, which promotes interactions between proteins that are necessary for proper assembly and function.

Typically, transmembrane signaling proteins are the transducers of the initial stimuli that set cellular pathways in motion. The signal transduction pathways in which the transmembrane signaling events are a part, are critical for generating responses to broad range of external stimuli that are generally recognized to be generated either by the organism itself (hormones, growth factors, other cells) or from foreign entities (foreign cells or cells recognized as foreign, viruses, bacteria, other pathogens and pathogenic materials, and allergens). Transmembrane signaling and signal transduction pathways are also indispensable for communication among cells in multicellular organisms. Consequently, almost all processes critical to the growth and function of multicellular organisms depend on transmembrane signaling. When these communication networks fail to execute an instruction, or when signaling becomes deregulated, diseases result, such as, for example, cancer, diabetes, and obesity. To illustrate the crucial role of cell signaling in disease, it has been reported that greater than 60% of all drugs, including drugs available in the marketplace and drugs that have been selected for market, target proteins involved in signal transduction pathways. With an estimated annual spending on early stage drug screening in excess of one billion dollars, there is a great need for innovations that improve the efficiency and accuracy of such screening assays.

“Transmembrane receptors” are key protein elements in the process of signal transduction. The receptors often span the membrane bilayer one or more times in order to convey information across it during the process of transmembrane signaling. It is widely known that membrane receptors interact with one another by clustering together in the membrane to form dimers, trimers, or more generally oligomers, and that the process of clustering and/or the formation of multimers is an integral part of the transmembrane signaling process. Dimers, are often generated through the association of two identical protein molecules to form homodimers, but heterodimers can form in other instances, through the specific association of two different receptors (See, e.g., Martin and Wesche, 2002; Bazan-Socha et al. 2005; Penuel et al., 2001). More generally hetero-oligomeric complexes form to orchestrate the transmembrane signaling. (See, e.g., Alarcon et al., 2003). Also, additional proteins involved with the process of transmembrane signaling have been reported to associate with the inner leaflet of the membrane through specific interactions with the receptor and/or the membrane itself. (See, e.g., Pawson and Nash, 2003). These too are part of the process of signal transduction.

Genome sequencing projects have produced a wealth of information that have brought about significant advances in descriptive cellular and molecular biology, including the establishment of familial and evolutionary classifications of a multitude of transmembrane receptors. (See, e.g., Ben-Shlomo et al., 2003). These works, along with the continuing efforts to determine the structures and functions of transmembrane receptors, have, altogether, led to the identification of unifying principles in the processes of transmembrane signaling, principles that are inextricably associated the special properties of the cell membrane.

Significant resources and attention have been devoted to the study of membrane-associated proteins; however, membrane samples of the proteins that are used in such biochemical experiments are frequently isolated from cells expressing the receptor at elevated levels, which can result in complex and heterogeneous samples. Also, receptor reconstitution is labor-intensive, and the conditions that maintain a high level of activity while also preserving the vectoral and lateral organization required for function can be difficult to find. Notably, it is the very association of receptors with membranes that invariably requires the use of detergent for the purification of receptors, which leads to well-known difficulties, including low yield and the disruption of critical protein-protein interactions. Low yields are typical and represent a major impediment to widespread use of such receptors in cell-free assay systems. Also, the solubilizing activity of detergents, which is the basis of their usefulness in other applications, such as membrane protein purification, represents a significant disadvantage in functional assays where protein-protein interactions are necessary. In this setting, detergents disrupt necessary interactions between the receptors in the membrane, as well as the interactions between receptors and receptor-associated proteins, and protein-protein interactions in general. While formulations of detergent compatible with functional activity can sometimes be achieved, these are identified only by time-consuming and case-specific methods, and the level of activity usually achieved often remains less than satisfactory.

To overcome these difficulties, researchers have attempted to identify key regions of the membrane-associated proteins that can be cloned out for study in vitro. Some of these receptor fragments support activity and have been commercialized for the study of pair-wise interactions, such as, for example, interactions between a protein domain that possesses enzymatic activity and a substrate. Much information has been lost in these situations, however, as signaling proteins are studied in environments that differ significantly from their natural, cellular environments.

It is apparent from the above that there is a continuing need for advancements in the relevant field, including new methods and materials for restoring function to membrane-associated proteins outside their natural, cellular environment. The present application addresses this need.

### SUMMARY

Using template-directed assembly of proteins, protein fragments and/or variants thereof, the present application provides methods and materials and/or complexes useful to analyze the functionality of membrane-associated polypeptides, such as, for example, portions of transmembrane proteins, membrane-associated proteins, and other proteins that bind to transmembrane proteins and membrane-associated proteins, and to analyze the effect of spatial organization on the functionality of the polypeptides. The methods and materials described herein provide a more native-like environment for analyzing the functionality of membrane-associated proteins, and thus provide effective tools for studies involving the detection of the level of enzyme activity of such proteins in an environment that closely resembles the native environment in the cell. This in turn provides a wide variety of useful applications, such as, for example, efficient processes for analyzing how drugs, drug candidates or other active agents affect the functionality of a membrane-associated protein, fragment, or pathway.

In one aspect, the invention can provide a means to assemble membrane-associated polypeptides through the use of a templating material for the purpose of generating associations among polypeptides, and providing a template/polypeptide complex that has a functionality correlating to the functionality of membrane-associated polypeptides present in the native environment. The present application, in its various embodiments, can be used with components derived from a wide variety of membrane-associated protein systems, such as, for example, signaling systems that require interactions among one or more like or unlike entities at the membrane surface to activate or enhance the biological function of the components. Reported herein are multiple examples corresponding to a large variety of human membrane-associated protein systems, showing how the assembly of selected fragments thereof (polypeptides) on a two-dimensional fluid membrane-like template restores functionality to a level much closer to native levels and manners of functionality, compared to that which can be achieved in solution or dispersion. While it is not intended that the subject matter of this application be limited to any theory, it is believed that functionality in various different systems can result from (1) an orienting effect produced by assembling proteins with a template, (2) the interactions that develop through the assistance of the template to facilitate clustering of the receptor proteins or other polypeptides to form dimers, trimers and more generally oligomers, and/or (3) the recruitment of associated signaling proteins or other reagents, which are altogether referred hereto as “signaling teams” (e.g., without limitation, one or more mixtures or combinations of polypeptides all coupled or linked directly to a template, one or more polypeptide of such a mixture/combination coupled/linked directly to a template and/or one or more polypeptides linked or coupled indirectly by association with one or more polypeptides that are linked to a template directly.) The broad utility of the methods and materials described herein is shown by the successful assembly of several human membrane-associated protein systems onto templates as described herein. With respect to certain non-limiting embodiments, the recombinant protein reagents used in some of the experimental work reported herein are cytoplasmic domains derived from receptor-tyrosine kinases (RTKs), as well as non-receptor tyrosine kinases and non-receptor serine-threonine kinases. RTKs are a large class of transmembrane receptor proteins, which are widespread in species belonging to the eukaryotic kingdom, including humans. Representative members of the RTK class have been investigated. RTKs function in pathways linked to numerous diseases including obesity, cancer, diabetes and developmental defects. The success of these systems in providing a high level of functionality establishes that template-directed assembly of functional protein fragments and/or signaling molecules can be reliably and predictably reproduced for a wide variety of membrane-associated protein systems, even complex human systems and protein systems characteristic of other higher organisms, such as, for example, mammalian systems, on the basis of known similarities in the organization and mode of action of protein fragments derived therefrom. The high level of biological activity of the protein reagents achieved in accordance with the application is representative of a wide variety of membrane-associated protein systems of medical relevance. The present application therefore describes a significant advancement in biomedical research, and provides methods and materials that are useful in a wide variety of protocols including, for example, protocols used to screen for drugs and candidate drugs that have an effect on a selected protein or protein system.

In one aspect of the invention, there is provided a method for analyzing in vitro the effect of a molecule upon an enzyme-catalyzed reaction or cascade. Such a method can comprise: (1) providing an aqueous fluid medium including one or more reagent; and a biologically active complex including a template and at least one polypeptide attached to the template, wherein the complex is functional under a given set of conditions to produce a measurable modification in the content of said one or more reagent or in said polypeptide; (2) introducing a test molecule, such as, for example, a drug, drug candidate, agonist or antagonist, into the fluid; and (3) measuring the modification to determine the effect of the test molecule on the reaction or cascade. The measurable modification can result from a wide variety of processes, such as, for example, the following: (1) a chemical modification to the polypeptide, or equivalently a protein or protein domain, resulting from intrinsic enzymatic activity of said polypeptide, protein or protein domain as it interacts with the template, (2) chemical modification of a soluble substrate reagent present in the fluid that is catalyzed by the polypeptide as it interacts with the template, (3) chemical modification of a soluble substrate reagent that is catalyzed by enzymatic activity of a signaling enzyme present in the fluid and recruited to the complex, (4) chemical modification to the polypeptide in a process catalyzed by a signaling protein that is recruited to the complex, and (5) chemical modification of a soluble substrate reagent present in the fluid that results from a reaction cascade initiated by the polypeptide as it interacts with the template or a signaling enzyme that is recruited to the complex. The measurable modification of a polypeptide can be, for example, phosphorylation, dephosphorylation, acetylation, methylation, acylation, glycosylation, glycosylphosphatidylinositol (GPI) anchoring, sulfation, disulfide bond formation, deamidation, ubiquitination, sumoylation, nitration of tyrosine, hydrolysis of ATP or GTP, activation of a fluorescent signal, release of a reaction product or utilization of a reagent initially present in the fluid. In one embodiment, the polypeptide comprises a receptor tyrosine kinase domain, and the process can comprise autophosphorylation of the receptor tyrosine kinase domain.

Without limitation, such a template can be a supported monolayer or a free-standing or supported bilayer, comprising a polar lipid. In one such embodiment, such a template can be a phospholipid vesicle. In other embodiments the template is a polymer vesicle, a polymer micelle, or a polymer molecule. In yet other embodiments, the template is coated onto a substrate material. Substrates can be, for example, glass slides, glass beads, silicon wafers, silicon chips, planar noble metals, colloidal noble metal, metal oxide layers, nanoparticulate materials, or polymer slabs, films or beads. In these examples the template can be, for example, a fluid phospholipid bilayer, a phospholipid monolayer or a polymer film.

In one embodiment, the polypeptide can be attached to a linker component effective to attach the polypeptide to the template. The linker component can be, for example, a component effective to covalently bond to the template, a component effective to interact with the template noncovalently by metal chelation, a component effective to interact with the template noncovalently by other complementary interactions, or an insertion domain effective to interact with the template noncovalently by insertion of at least a portion of the domain into the template. In an embodiment in which the linker component comprises a component effective to interact with the template noncovalently by metal chelation, the metal or metal ion can be associated either with the template or with the linker component prior to the interaction. In one embodiment, the linker component comprises a genetically engineered histidine tag. In an embodiment in which the linker component comprises an insertion domain, the insertion domain can be configured to interact with the template noncovalently by insertion of at least a portion of the domain into the template, wherein at least a portion of the insertion domain interacts with the template by hydrophobic interactions. In another embodiment, the linker is an insertion domain that comprises a genetically engineered peptidyl insertion domain. The insertion domain can alternatively comprise an anchoring moiety formed by the adaptation of naturally occurring mechanisms, such as, for example, palmitoylation, myristoylation, prenylation and geranylation or through a GPI linkage. In another embodiment the anchoring moiety can be comprised of a synthetic analog of these naturally occurring mechanisms of palmitoylation, myristoylation, prenylation, and geranylation or GPI linkage. In another embodiment, the linker component comprises an engineered amphipathic helix that has affinity for the surface of the template.

In another aspect of the invention, there is provided a method for determining the effect of one or more mutations on the functionality of a membrane-associated protein. It is well known to those knowledgeable of biology and medicine that amino acids substitutions, deletions, or insertions in proteins, which can be caused by mutations in the DNA from which the proteins are generated, can result in dramatic differences in the functionality of said proteins, such as for example, in a signaling pathway. Such method can comprise: (1) providing a control complex that includes a control (e.g., nonmutated) polypeptide, the control polypeptide comprising a membrane-associated protein, a fragment thereof or a variant thereof featuring substantially normal functionality, whereby the control complex is functional under a given set of conditions to produce a measurable modification in the content of one or more reagent or in said polypeptide, the control polypeptide modified to incorporate thereon a linker component that does not substantially affect the functionality of the control polypeptide, wherein the modified control polypeptide is attached to a template; (2) providing a test complex comprising a corresponding test polypeptide featuring one or more mutations; (3) contacting the test complex and the control complex in aqueous fluids to said one or more reagent under similar reaction conditions; and (4) measuring the modification for the test complex and the control complex, and comparing same to score the functionality of the test and control polypeptides.

In another aspect, the invention can provide a complex which can comprise (1) a template; and (2) a polypeptide linked to the template, the polypeptide comprising a human membrane-associated protein, or a fragment thereof, or—optionally—a polypeptide having at least about 80% identity thereto, the polypeptide having attached thereto a linker component that does not substantially affect the functionality of the polypeptide and that is effective to connect, link or couple the polypeptide to the template. In one embodiment, the polypeptide is derived from a transmembrane receptor protein. In another non-limiting embodiment, the polypeptide is a cytoplasmic domain derived from a receptor tyrosine kinase, a non-receptor tyrosine kinase or a non-receptor serine-threonine kinase. In alternative embodiments, the polypeptide comprises, for example, an insulin receptor protein, an ErbB4 receptor protein, an Axl receptor protein, an EphB2 receptor protein, a fragment thereof or a functional variant thereof.

In yet another aspect, the invention can provide a complex that can comprise: (1) a template; and (2) a polypeptide comprising a protein in its entirety or its fragment, that is not a transmembrane protein, but is a protein that functions via other types of interactions with a membrane. This aspect of the application contemplates a protein that is normally associated with the membrane under resting conditions, or a protein recruited to the membrane as the result of a change in conditions, that may, for example, result from a stimulatory event. In another embodiment the protein is a membrane associated non receptor tyrosine kinase or a serine-threonine kinase. Representative of the latter, as would be understood by those skilled in the art, the Akt proteins are members of the protein kinase B family and the AGC kinase group. The PDK proteins are also members of the AGC kinase group. (Without limitation, reference is made to Examples 4-6 and FIGS. 7-13, below.) In another embodiment the protein is a member of Src family of kinases, the Lyn kinase, or a Syk family kinase. All these protein kinases belong to a single superfamily whose catalytic domains are related in sequence and belong the typical kinase group. Furthermore, mTOR is a serine/threonine kinase that is a member of the atypical kinase group.

In yet another aspect, the invention can provide a complex that can comprise: (1) a template; and (2) a polypeptide having an N-terminal end linked to the template and a C-terminal end linked to the template, the N-terminal end and the C-terminal end of the polypeptide both modified to incorporate thereon a linker component that does not substantially affect the functionality of the polypeptide loop and that is effective to link the respective ends of the polypeptide to the template. In one embodiment, the polypeptide comprises a fragment of a multi-pass transmembrane protein. Such doubly anchored peptide may be derived, for example, from the cytoplasmic loops of G-coupled receptors and may serve, for example, to recruit the one or more components of the heterotrimeric G-proteins.

In yet another aspect of the invention, there is provided a complex that can comprise: (1) a template; and (2) a plurality of different polypeptides linked to the template, each of the polypeptides modified to incorporate thereon a linker component that does not substantially affect the functionality of the polypeptide and that is effective to link the polypeptides to the template. In one embodiment, the plurality of polypeptides comprises a plurality of fragments of a multi-pass transmembrane protein. The fragments can be, for example, cytoplasmic fragments or extracellular fragments.

In another aspect, the invention can provide a method for performing a manufacturing process that requires an assembly of one or more polypeptides on a template in a ‘team’, or an associated complex, which in this aspect of the application catalyzes the modification of a substrate in an effective manner. Examples of substrates contemplated by this aspect of the application include, for example, a constituent member of the assembled protein team, a portion of the template, or a reagent molecule that is separately included in the fluid in which the complex is suspended. The method comprises: (1) providing an aqueous fluid including one or more reagent; and a biologically active complex including a template and at least one polypeptide attached to the template, wherein the complex is functional under a given set of conditions to generate a reaction product that results from the functionality of the complex; and (2) isolating the reaction product. The method may also include the introduction of a substrate molecule, which may be part of the assembled team, part of the template, or a molecule added into the fluid separate from the complex, such as, for example, after the active complex is generated. In one embodiment, this aspect of the application may be applied to the synthesis of a protein that is modified post-translationally under the conditions of a team-assembled reaction. In another embodiment this aspect of the application may be used to synthesize phosphorylated receptor tyrosine kinase domains.

In still another aspect, the invention can provide a method for determining whether an observed disease state of a patient results from sub-standard functionality of a membrane-associated protein. The method includes: (1) providing a test complex that includes a test polypeptide isolated from a patient, the test polypeptide comprising a membrane-associated protein or a fragment thereof suspected to exhibit sub-standard functionality, the test polypeptide modified to incorporate thereon a linker component that does not substantially affect the functionality of the test polypeptide, the modified test polypeptide attached to a template; (2) providing a control complex comprising a corresponding control polypeptide featuring normal function, the control complex functional under a given set of conditions to produce a measurable modification in the content of one or more reagent or in said polypeptide under suitable reaction conditions; (3) contacting the test complex and the control complex in aqueous fluids to said one or more reagent under similar conditions; and (4) measuring the modification for the test complex and the control complex and comparing same to score the functionality of the polypeptide on the reaction or cascade.

Further embodiments, forms, features and aspects of the present application shall become apparent from the detailed description and figures provided herewith.

### BRIEF DESCRIPTION OF THE DRAWINGS

FIG. 1.

A: Proteins, protein domains or other protein fragments typically have little tendency to organize in solution.

B: The template facilitates the assembly of one or more kinds of proteinaceous entities into functional units. An interaction between the template and the proteins localize them at the surface and orients them; this promotes the formation of functional units, e.g. dimers, or more generally oligomers. Generally, but not exclusively, interactions between the template-assembled entities are essential for function.

C: Mixtures of proteins, protein domains and other protein fragments, which have functional activity in an assembled form often have little tendency to organize in solution. Without the full complement of interactions, the protein mixtures do not display the essential biological function.

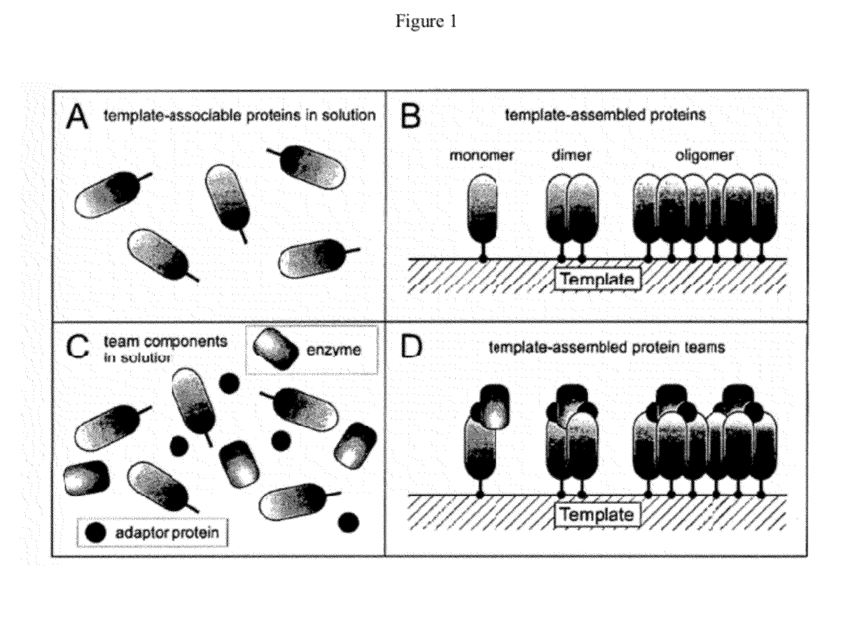

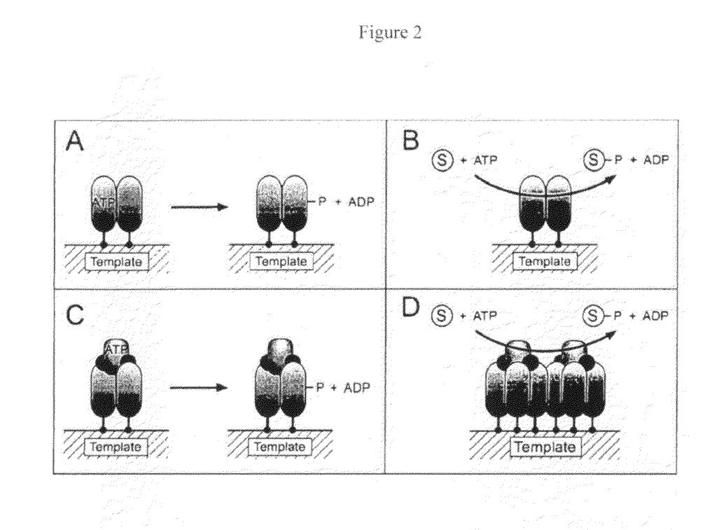

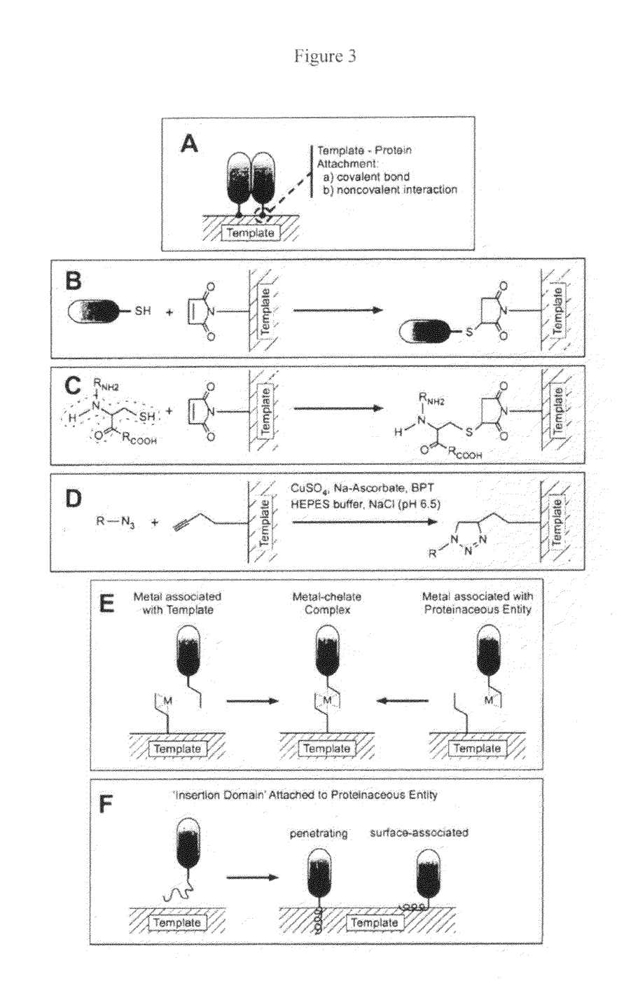

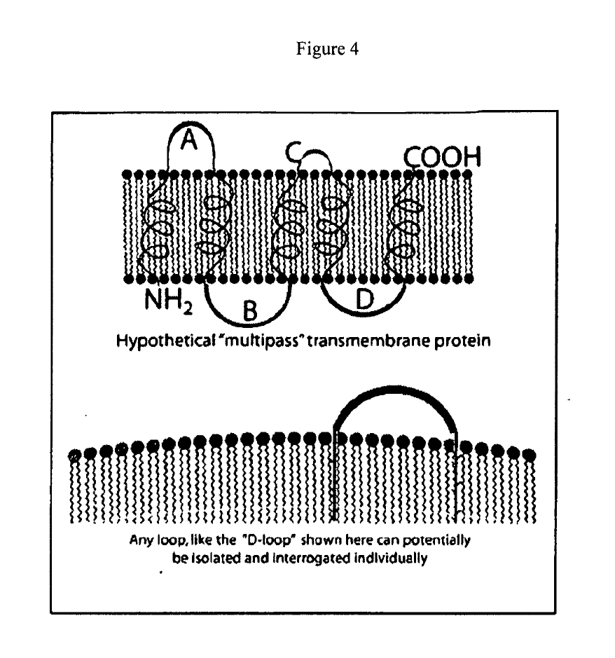

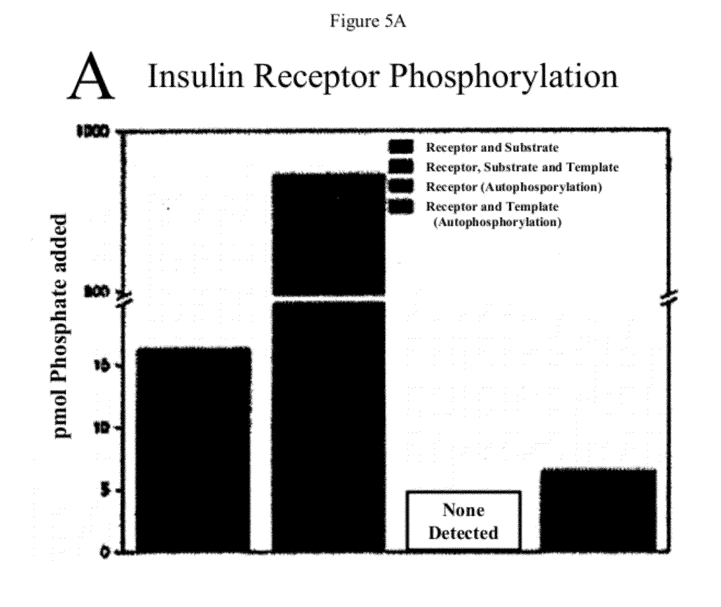

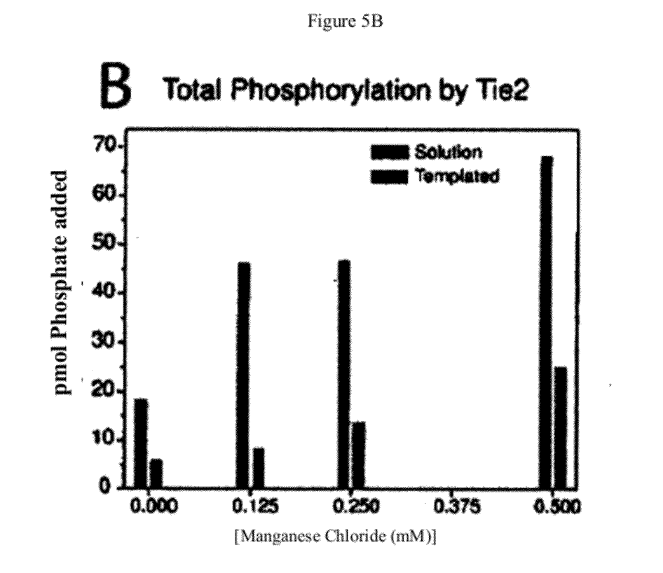

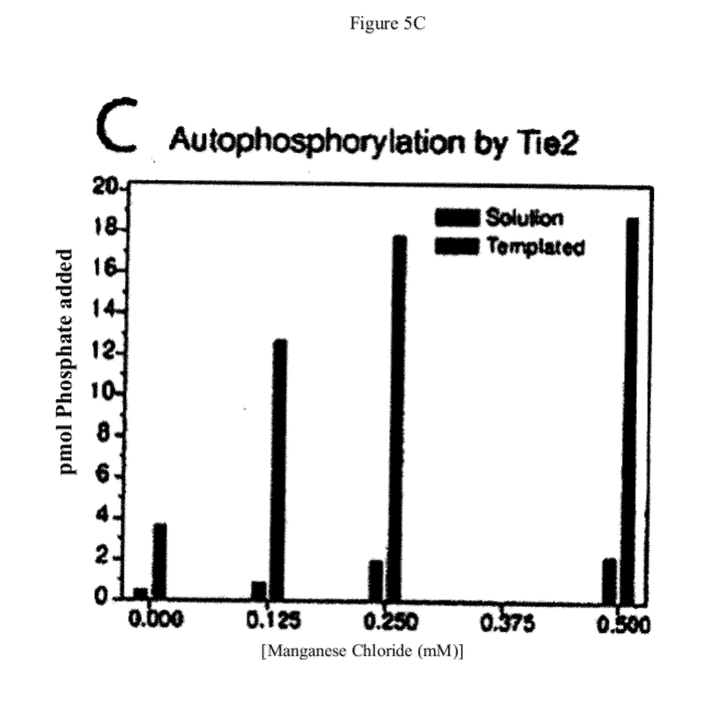

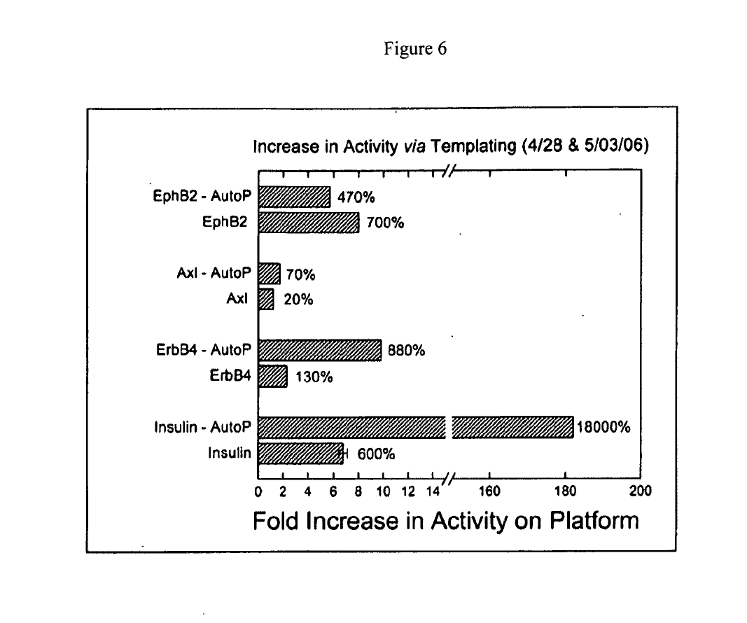

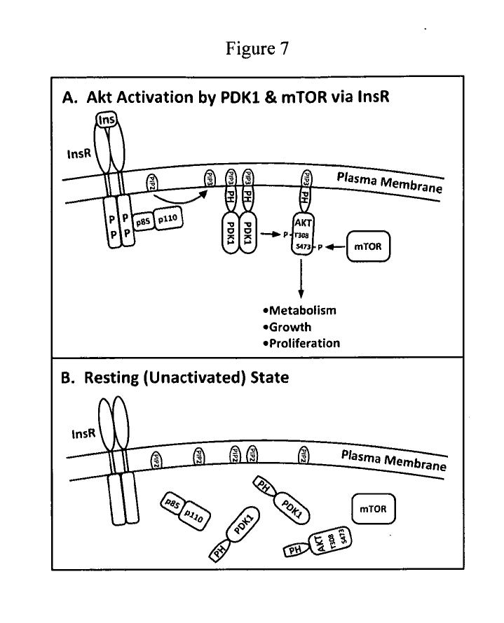

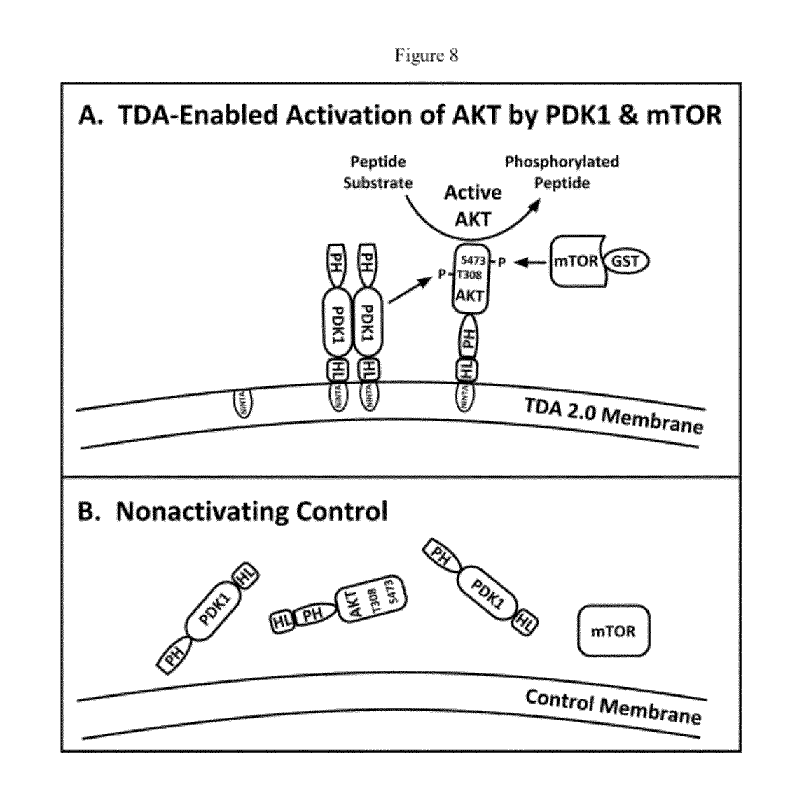

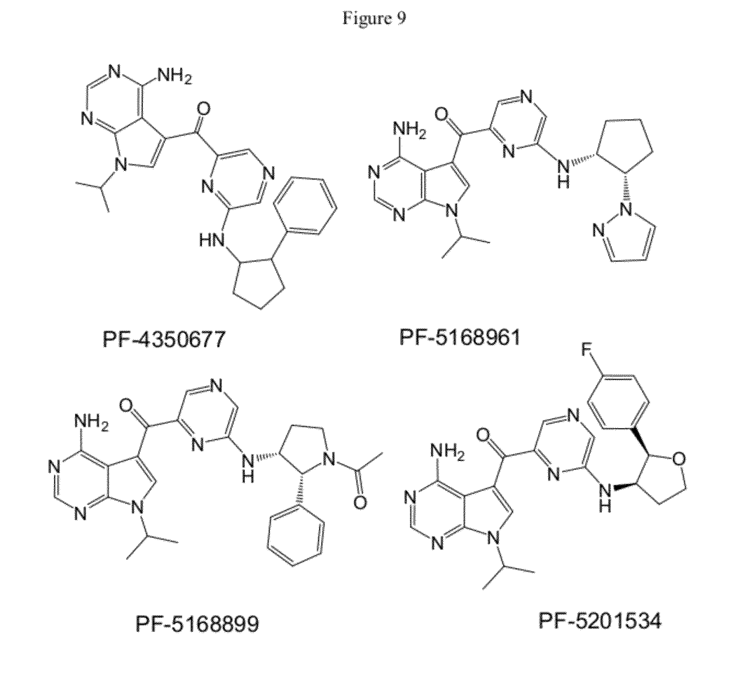

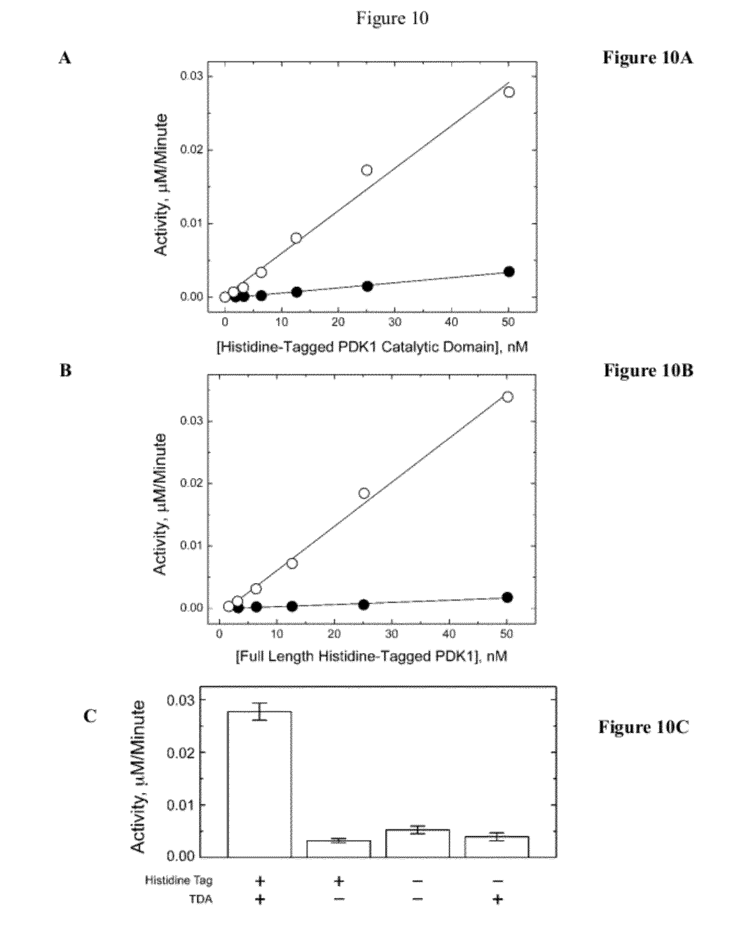

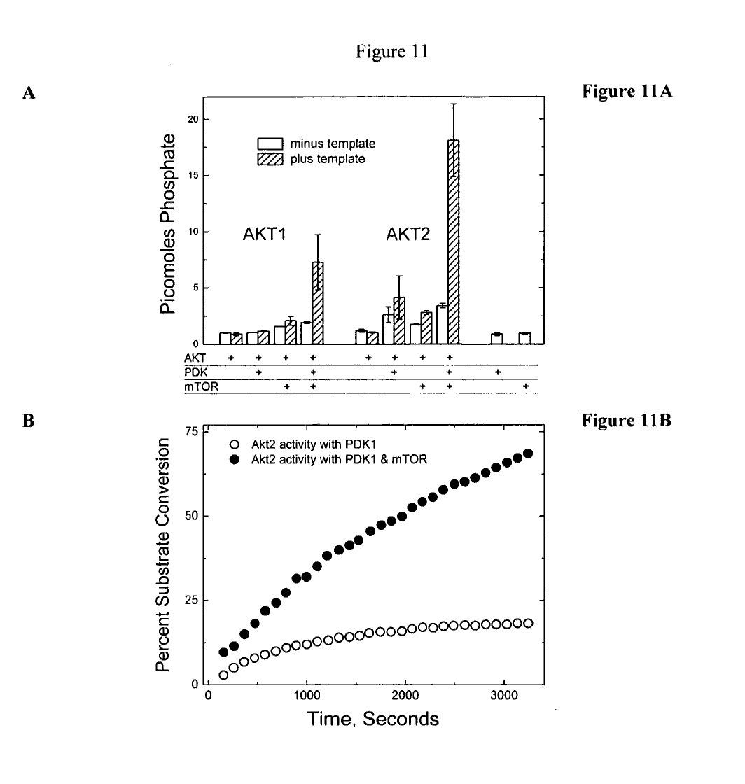

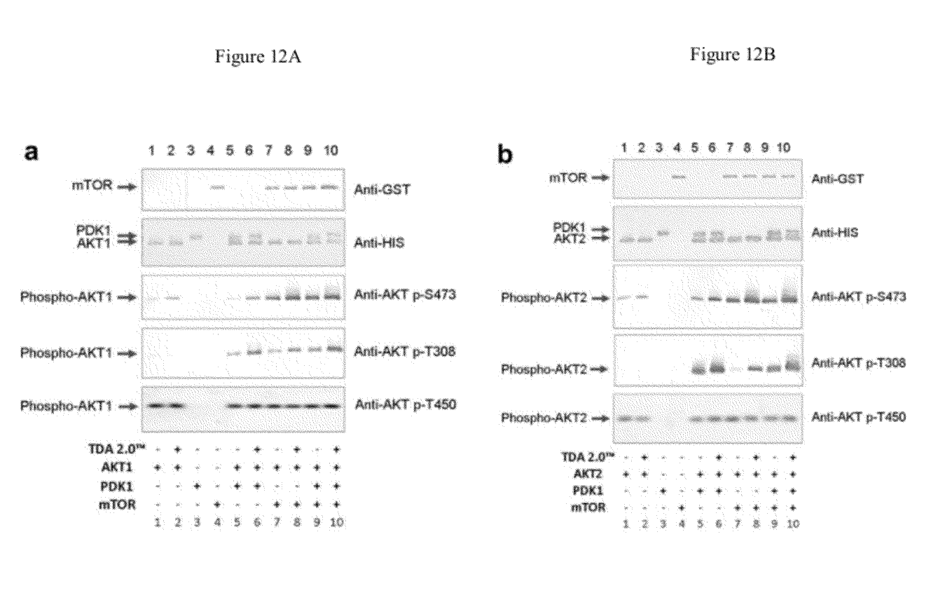

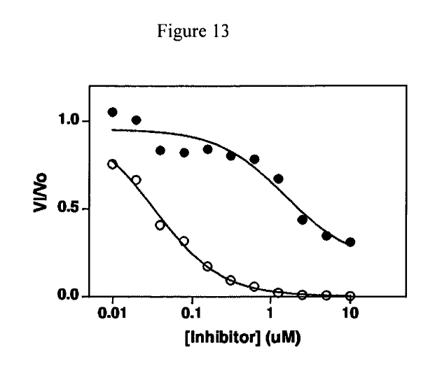

D: The template facilitates the assembly of a mixture of protein domains, adaptor proteins, signaling enzymes and other proteinaceous entities into functional units. One or more proteinaceous entities in the mixture interact specifically the template (these components are ‘template-associable’ as in FIG. 1A), which form an assembly that recruits adaptor proteins, and other signaling proteins, to form a functioning assembly.

FIG. 2. Representative Embodiments

A: This figure represents an embodiment of the application applied to receptor tyrosine kinases (RTKs) and serine-threonine kinases. This embodiment assembles a receptor signaling domain that has autocatalytic capability in which it binds co-substrate, for example adenosine tri-phosphate, ATP, and catalyzes its own chemical modification, for example in the transfer of a phosphate group from ATP to form phosphorylated protein and adenosine di-phosphate, ADP.

B: This figure illustrates another embodiment of the application involving template-assembled receptor signaling domains, including those derived from RTKs, to catalyze a chemical reaction between two substrates, for example ATP and a phosphate-accepting substrate (S), to generate products, for example phosphorylated substrate (S—P) and ADP.

C: An embodiment of the application involving a template-assembled protein team, the formation of which is illustrated by FIGS. 1C and D. A signaling enzyme is recruited to the signaling team through interactions with the template-associated entity and/or an adaptor protein. The signaling enzyme binds substrate, for example ATP, as shown here, and catalyzes the chemical modification of the template-associated entity, for example, as in a phosphorylation reaction.

D: Another embodiment of the application involving a template-assembled protein team, which illustrated in FIG. 1D. A signaling enzyme is recruited to the signaling team through interactions with the template-associated entity and/or an adaptor protein. The signaling enzyme binds two substrates to catalyze the chemical modification of one substrate, for example the transfer of a phosphate group from ATP to the substrate, S, to generate phosphorylated substrate S—P and ADP.

FIG. 3. Modes of Interaction with the Template

A: The proteinaceous entity has a point of attachment to the template, which can be generated either through the formation of a covalent bond between the proteinaceous entity and the template, or through a noncovalent interaction between the proteinaceous entity and the template.

B: A specific example of covalent attachment to the template in which a sulfhydryl group on the protein reacts with a maleimide moiety on the template.

C: A cysteinyl residue (encircled) within the polypeptide chain of a protein, a protein domain, a protein fragment or a peptide reacts with maleimide moiety to generate a covalent thioether linkage. R_{NH2} and R_{COOH} represent portions of the protein, protein domain, protein fragment or peptide that are N-terminal and C-terminal, respectively, to the cysteinyl residue.

D: Covalent attachment using the copper-catalyzed coupling of alkyne and azide groups. Prior to coupling, the azide moiety resides within the proteinaceous entity and the alkyne part of the template. In another embodiment (not shown), the alkyne moiety may reside within the proteinaceous entity and the azide moiety is attached to the template. BPT: bathophenanothroline disulfonate.

E: An illustration of a noncovalent metal-chelate-assisted interaction between the proteinaceous entity and the template. A metal or metal cation (M) is associated either with a moiety attached to the template (left) or a moiety attached to the proteinaceous entity (right). In both embodiments, M provides for a bridging interaction between the associated entity and the template (middle).

F: A generic illustration of noncovalent interactions between the proteinaceous entity and the template by way of an ‘insertion domain’. Insertion domains are part of the proteinaceous entity that can form a noncovalent association with the template, either by penetrating into the template, by associating with the template surface, or by a combination of penetration and surface association.

FIG. 4. Multipoint Template Attachment

A: An embodiment of the application in which the template-associable entity has more than one point of attachment to the membrane. An example of a multipass receptor protein, illustrated at the top of FIG. 4, has the extramembranous loops A, B, C and D between transmembrane segments. The lower part of FIG. 4 depicts a proteinaceous entity associated with the template at two points.

FIG. 5. Examples of Enhanced Activity with Template-Assembled Receptor Tyrosine Kinases

A: Autophosphorylating and substrate-phosphorylating activities of the Insulin Receptor RTK domain. (Upstate product number 14-553). The substrate in the assay is Axltide, from Upstate (product number 12-516), which is a peptide of composition KKSRGDYMTMQIG

Activities in solution and on templates, either with or without substrate:

1. Left-most Column: Insulin RTK domain plus exogenous substrate (Axltide) in solution (no template).

2. Second Column from Left: Insulin RTK domain plus exogenous substrate (Axltide) in the presence of template.

3. Second Column from Right: Insulin RTK domain (no exogeneous substrate) in solution.

4. Right-most Column: Insulin RTK domain (no exogeneous substrate) in the presence of template.

B: The autophosphorylating activity, measured as pmol of acid precipitatable phosphate at 10 min., of the Tie2 RTK domain plus the substrate-phosphorylating of the Tie2 RTK domain. The histogram shows the dependence of the activity on MnCl₂ concentration. Results of measurements at each concentration of MnCl₂ are provided by a pair of bars, the left bar of each pair representing the activities measured in solution and the right bar of each pair representing the activities measured in the presence of template. The Tie2 RTK domain is an Upstate (product number 14-540), and the substrate is poly([Glu₄Tyr) from Sigma-Aldrich (product number P7244).

C: The autophosphorylating activity of the Tie2 RTK domain, measured as pmol of acid precipitatable phosphate at 10 min., either in solution (left bar of each pair) or in the presence of template (right bar of each pair). The Tie2 RTK domain is an Upstate reagent, product number 14-540. The histogram shows the dependence of the activity on MnCl₂ concentration.

FIG. 6. Fold Increase in Phosphorylation Activity Produced by Template Directed Assembly of Selected Tyrosine Kinase Domains

FIG. 7. PI3K pathway activation, using the Insulin Receptor (InsR) as an example. (A) The activation steps in the PI3K pathway are depicted. Insulin (Ins) binding to the Insulin receptor (InsR) stimulates receptor auto-transphosphorylation on specific tyrosines residues in the cytoplasmic domain of the receptor dimer. These phosphotyrosines are binding sites for phosphatidylinositide-3 kinase, PI3K (p85/p110), which by recruitment to the membrane catalyzes the transformation of membrane lipids, such as phosphatidylinositide-(2,3,4)-trisphosphate (PIP3) from phosphatidyl inositide-(2,3,4)-trisphosphate (PIP2). PIP3 recruits 3-phosphoinositide dependent protein kinase-1 (PDK1) and Akt to the membrane through an interaction between a Pleckstrin Homology (PH) domain and PIP3. Membrane-associated PDK1 activity increases, possibly by auto-transphosphorylation of a PDK1 dimer, as depicted in the figure. PDK1 and the mammalian Target of Rapamycin (mTOR) both phosphorylate Akt, on threonine-308 (T308) and serine-473 (S473), respectively, and thereby activate it. Phosphorylation of Akt substrates ultimately leads to biological effects, such as, but not limited to lipolysis, glucose update, and growth or proliferation. (B) Without Ins, the signaling pathway is in a resting (unactivated) state: InsR is not phosphorylated and thus neither PDK1 nor AKT are recruited to the membrane, where activation occurs. Also, phosphatases (not depicted) dephosphorylate enzymes in the PI3K pathway, which contributes to the maintenance of the unactivated state.

FIG. 8. Replicating the activation of Akt by PDK1 and mTOR using TDA 2.0™ template membranes. (A) Histidine-tagged constructs of Akt and PDK1 deliver these enzymes to the template membrane, which contains the nickel salt of the lipid 1,2-dioleoyl-sn-glycero-3-[(N-(5-amino-1-carboxypentyl)iminodiacetic acid)succinyl] (NiNTA). The interaction of Akt and, as shown here, PDK1, with the membrane are promoted by the terminal histidine-tagged linkers (HL) in the respective proteins. The HL moiety tethers these proteins to the template via the NiNTA—His-tag interaction. Furthermore, to demonstrate the extended utility of this assay format as a functional multicomponent assemblage, GST-tagged mTOR is included, which phosphorylates Akt on serine 473 (S473). When Akt is phosphorylated on both 5473 and T308, Akt kinase activity is increased substantially. Other engineered constructs, not depicted, can elicit Akt activation, provided that Akt becomes associated with the membrane through a membrane tether, such as the HL-NiNTA interaction. (B) Nonactivating controls can be used to demonstrate the necessity of the Akt-membrane interaction, for example by, but not limited to, the preparation of a control membrane template that lacks the NiNTA lipid, which is unable to assemble Akt on the template and thus fails to activate Akt.

FIG. 9. 2D structure of carbonyl-4-amino-pyrrolopyrimidine (CAP) inhibitors (e.g., drug and drug candidate test molecule). All the compounds used in this study are enantiomers except for PF-4350677, which was a racemic mixture.

FIG. 10: The presence of TDA 2.0™ liposomes in the reaction media boosts the activity of PDK1 activity. As illustrated in FIG. 10A, the activity of the catalytic domain of PDK1 (KD-PDK1) alone increases by 4-5 fold in the presence of these lipid based vesicles (∘) as compared to the reaction in the absence of it (). A greater effect (˜20-fold) was observed in the presence of full length-PDK1 (FL-PDK1) when mixed with the same lipid (∘) as compared to the reaction in the absence of it () (FIG. 10B). As a control study, FIG. 10C shows that the increase of PDK1 enzyme activity can only be observed when the enzyme possesses a His tag. The last experiment was conducted in the presence of 50 nM of enzyme while peptide, TDA 2.0™ and ATP concentrations are similar to the rest of this study (see experimental section).

FIG. 11. The presence of TDA 2.0™ liposomes in the reaction media boosts the activity of AKT activity when combined with PDK1, mTOR in a cascade assay. (A) Results of the radiometric filter binding assay. Production of the phosphorylated Crosstide™ peptide, an Akt substrate, was determined in the absence (open columns) or the presence (striped columns) of template (TDA 2.0™), and either without PDK1 and mTOR, with one or the other, or with both PDK1 and mTOR. (B) Results of a Caliper-based assay. The combination of PDK1 and TDA 2.0™ have a limited impact upon the activity of AKT2 (∘), while the addition of mTOR to the mixture significantly boosts the stability and the activity of AKT2 (). Similar effect was observed with AKT1 too (data not shown).

FIG. 12: Western blot analyses examining the phosphorylation state of Akt1 (A, left panel) and Akt2 (B, right panel) after kinase assays were performed in various combinations of PDK1, mTOR and TDA 2.0™. The anti-His-tag (Anti-HIS) and anti-GST antibodies were used to verify the integrity and concentration of added protein (Akt, PDK1, mTOR) in the sample aliquots. The anti-phospho-threonine and anti-phospho-serine antibodies were used to assess the extent of phosphorylation on Akt.

FIG. 13A-B: Inhibition of PDK1 and AKT1 by PF-5168899. Inhibition of PDK1 by PF-5168899 produced a Kiapp in the nM range (∘). Downstream inhibition by PF-5168899 of AKT1 activity, in a cascade assay with PDK1 and AKT1, had a limited effect and produced a Kiapp in the μM range ().

### DEFINITIONS

Axltide. Is an example of an oligopeptide that has the specific sequence KKSRGDYMTMQIG. K is the one letter abbreviation for the amino acid lysine, S is the one letter abbreviation for the amino acid serine, R is the one letter abbreviation for the amino acid arginine, G is the one letter abbreviation for the amino acid glycine, D is the one letter abbreviation for the amino acid aspartate, Y is the one letter abbreviation for the amino acid tyrosine, M is the one letter abbreviation for the amino acid methionine, T is the one letter abbreviation for the amino acid threonine, Q is the one letter abbreviation for the amino acid glutamine, and I is the one letter abbreviation for the amino acid isoleucine. Thus, KKSRGDYMTMQIG, represents an oligopeptide that consists of H₂N-lysine-lysine-serine-arginine-glycine-aspartate-tyrosine-methion-ine-threonine-methionine-glutamine-isoleucine-glycine-COOH, where H₂N and COOH are used to denote the amino and carboxy termini of the oligopeptide, respectively. Axltide, as defined here, is a model substrate of the Axl receptor tyrosine kinase and the insulin receptor tyrosine kinase.

Poly([Glu₄Tyr)ₙ. A synthetic polypeptide comprised of repeating units of “glutamate-glutamate-glutamate-glutamate-tyrosine” of the form H2N-(glutamate-glutamate-glutamate-glutamate-tyrosine)ₙ—COOH, where n is meant to signify the number of the repeating units that are joined together, and typically has a value between 4 and 30.

Polypeptide. Polypeptides are polyamide polymers, which typically, but not always, consist of two or more amino acids of the L-enantiomeric form of alpha amino acids. Variations include, but are not limited to the D-enantiomeric forms of alpha amino acids and amino acids with unnatural side chains. It is the intention of this definition to include naturally occurring proteins and proteineacous entities. In addition the definition is intended include materials that are not considered to be fully functional proteins, such as for example peptides, oligopeptides and hybrid molecules of which polypeptides constitute only a part. Polypeptides can be generated by (1) chemical synthesis, (2) in vitro translation, (3) in vivo synthesis through the use of protein engineering and molecular biology, or (4) isolation from naturally occurring sources.

Protein. Typically, but not exclusively refers to a polypeptide of natural origin. A protein is typically but not exclusively of sufficient length to adopt well-defined tertiary structure. The terms protein, protein domain, and protein fragment are used interchangeably. Proteins that possess catalytic activity are enzymes.

Team. A signaling team is typically, but not limited to, proteins and protein fragments that function together in a way that the individual elements, of which the team consists, could not. In preferred embodiments the elements of the team are proteins, protein domains and polypeptides. In other embodiments of the application the elements of the team can also include lipids, carbohydrates, nucleic acids and other prosthetic groups that are either covalently or noncovalently associated with protein components of the team.

Template. A template is a molecular entity, either naturally occurring, synthetic or a hybrid of natural and synthetic parts, which facilitates functional interactions between the participating elements of a biochemical process. In one embodiment, the participating elements of a biochemical process are proteins and protein fragments that function together in cellular signal transduction pathways. In one embodiment, the template is a phospholipid membrane, arranged as a liposome, which has elements—such as a fluid phase, depending on molecular components used—that facilitate the assembly of the participating elements.

With reference to the figures and accompanying discussion and examples, abbreviations used herein include: DOPC, 1,2-dioleoyl-sn-glycero-3-phosphocholine; DOGS-NTA, 1,2-dioleoyl-sn-glycero-3-{[N(-amino-1-carboxypentyl)-iminodiacetic acid]-succinyl}ammonium salt); DOGS-NTA-Ni²⁺, DOGS-NTA Nickel Salt; SUV, small unilamellar vesicle; LUV, large unilamellar vesicle; Ni-NTA, nickel-nitrilotriacetic acid; ATP, adenosine triphosphate; ADP, adenosine diphosphate; GTP, guanosine triphosphate; GDP, guanosine diphosphate

### DETAILED DESCRIPTION

For the purposes of promoting an understanding of the principles described herein, reference will now be made to the embodiments set forth herein and specific language will be used to describe the same. It will nevertheless be understood that no limitation of the scope of the present application is thereby intended. Any such alterations and further modifications in the described devices, systems, processes and methods, and such further applications of the principles described herein are contemplated as would normally occur to one skilled in the art to which this application relates.

The present application provides methods and materials involving analytical processes and manufacturing processes that require in vitro activity of membrane-associated polypeptides, such as, for example, portions of transmembrane proteins, membrane-associated proteins, variants thereof, and other proteins that bind to transmembrane proteins and membrane-associated proteins in vivo. One application of the methods and materials described herein involves the use of functional polypeptides in vitro to study the effect of one or more active agents on the functionality of the polypeptides, for example during screening protocols for assessing the efficacy of large numbers of drug candidates with respect to a given membrane-associated protein system. The application provides a native-like environment in vitro for performing the functionality analysis, and thus provides effective tools for studies involving the detection of enzyme activity levels in an environment that closely resembles the native environment of a cell. Another application of the methods and materials described herein involves the assembly of functional proteins in vitro for the manufacture of reagents that require the use of a reaction cascade involving functional membrane-associated proteins, or that can be more economically achieved using such proteins. As described herein, a homogeneous or heterogeneous template can be used to assemble polypeptides, and optionally additional reagents, to provide a functional complex exhibiting the biochemical activity of a membrane-associated protein or protein system, such as, for example, a signaling pathway. The complex can be used to analyze the effect of an active agent on the functionality of the protein or protein system, to analyze the effect of a mutation on the protein or protein system, or to produce reaction products of value in a novel manufacturing process.

The templates described herein provide a synthetic environment that mimics organization and asymmetry inherent in cell membranes, which creates an environment in which receptor proteins can exhibit their native functionality (such as, for example, effectively convey information between the inside and outside of the cell), and in which other types of proteins can more readily assemble for other types of function than could occur in solution. The reduction in the degrees of freedom experienced by transmembrane and peripheral membrane proteins provides a strong driving force for lateral organization, which can be essential for function, e.g. ligand-induced clustering.

Membrane-associated proteins often need other ‘team members’ organized in the appropriate fashion to become fully functional. This activity typically arises from the assembly of a complex of proteins, which is referred to herein as ‘a signaling team’ that forms on or near the membrane surface. This illustrates and underscores the fundamental flaw in the prior attempts to restore functionality of membrane-associated proteins in vitro. The membranous environment provides the necessary chemical setting to assemble large teams of proteins for biologically relevant signaling. As depicted in FIG. 1 this cannot be achieved by analysis of single proteins interacting with a single partner in solution as these proteins have lost the two-dimensional information afforded by the membrane surface.

In accordance with the present application, template-directed assembly methods and materials are used to provide functional complexes in vitro of such membrane-associated protein systems in which complexes of multiple components, either transient or stable, are required for activity. The binding of a protein or suitable protein fragment to a template promotes a lateral organization among components, such as, for example, cytoplasmic fragments of transmembrane signaling proteins, that resembles the organization of cytoplasmic domains in the receptor-containing membranes of cells.

A functional protein complex in vitro provides two desired features. First, the complex exhibits activity measurably greater than would the same reagents in solution without a template (i.e., activity closer to the activity of the corresponding membrane-associated protein system in vivo). Here, greater activity may, in addition, refer to a more consistent regulation of a biological functionality similar to that which is observed in the cell, and therefore does not simply mean an increase in the magnitude of an enzyme activity. Second, the activity of the complex results in a measurable change to a test sample when the complex is placed in a solution containing the reagents necessary for the complex to function. As used herein, the phrase “measurably greater” is used to indicate that the complex has an activity at least about fifty percent greater than the activity of the same reagents in solution without a template, as measured by various methods that are known to those skilled in the art. For example, progress toward the completion of a reaction, or the rate of product formation as in the generation of post-translationally modified polypeptide, are two such examples. Here, post-translationally modified polypeptide can be taken to mean the phosphorylation of amino acid side chains, most typically on tyrosine, serine or threonine. More generally it should be evident that numerous other products of biochemical reactions can be measured, for example, but not limited to, protein phosphorylation, dephosphorylation, ATP hydrolysis, GTP hydrolysis, acylation, ubiquitination and methylation.

The diversity of signal transduction pathways gives rises to numerous embodiments that use a template to achieve functional interactions, which would not emerge with the isolated components in solution. The term “template” is used to refer to a material or an agent that facilitates the creation of relevant functional interactions. FIG. 1 is provided to illustrate the function of a template, but at the same time the illustrations of template in FIG. 1 are in no manner meant to confer a specific geometry, topology or structure to the template. FIG. 1B illustrates the generation of functional interactions by recruiting one or more proteinaceous entities from solution (FIG. 1A) through an association with the template. Interactions, for example, include homo- and hetero-dimer, trimer and oligomer formation among the template-associated species. FIG. 1D illustrates the manner in which a ‘team’ or ‘complex’ of proteins form through the assistance of a template, from a mixture of unassociated or partly associated proteins in solution (FIG. 1C), through the association of one or more kinds proteinaceous entities with the template, one more kinds of adaptor proteins and one or more membrane-associated proteins, which are recruited through an interaction with the template-associated entities. These recruited entities may or may not possess an intrinsic enzymatic activity.

In one embodiment, the measurable change results from a chemical modification to the polypeptide resulting from intrinsic enzymatic activity of the polypeptide as it interacts with the template. For example, in one embodiment, the polypeptide has intrinsic enzymatic activity, which leads to its chemical modification upon being attached to the template under suitable conditions. An example of this type of modification is the autophosphorylation, or more precisely an autophosphorylation reaction that occurs in trans, of receptor tyrosine kinase (RTK) domains (RTKs), as depicted in FIG. 2A. In another embodiment, the measurable change results from a chemical modification of a soluble substrate reagent present in the fluid that is catalyzed by the polypeptide as it interacts with the template, as depicted in FIG. 2B. This embodiment is exemplified by template-assembled RTKs that, with ATP, phosphorylate an added substrate molecule (depicted by the letter ‘S’ in FIG. 2B), such as for example poly([Glu₄Tyr) or a short peptide such as ‘Axltide’ (KKSRGDYMTMQIG), where these substrate molecules become phosphorylated on the tyrosine (Y) residues. In yet another embodiment, the measurable change results from a chemical modification of a soluble substrate reagent that is catalyzed by enzymatic activity of a signaling enzyme present in the fluid that is recruited to the complex, as depicted in FIG. 2C. In still another embodiment, the measurable change results from a chemical modification to the polypeptide in a process catalyzed by a signaling protein that is recruited to the complex, as depicted in FIG. 2D. In still yet another embodiment, the measurable change results from a chemical modification of a soluble substrate reagent present in the fluid that results from a reaction cascade initiated by the polypeptide as it interacts with the template or a signaling enzyme that is recruited to the complex. A wide variety of membrane-associated protein systems are known that function in these exemplary ways and in a variety of other ways, and are contemplated by the present application. The application is not limited to a specific mechanism of protein action, but rather encompasses a wide variety of mechanisms that are achievable upon attachment of the polypeptide to a template that mimics the properties of the membrane environment.

As stated above, for the use of a complex to provide practical and useful information, the changes in the composition of the aqueous fluid in which the complex is contained that result from the activity of the assembled polypeptides, and other reagents when present, must be measurable. Examples of the types of changes that are readily measurable to provide useful information regarding the functionality and/or activity of the complex can include protein modifications, such as, for example, phosphorylation, acetylation, methylation, acylation, glycosylation, glycosylphosphatidylinositol (GPI) anchoring, sulfation, disulfide bond formation, deamidation, ubiquitination, sumoylation, and nitration of tyrosine. Alternatively, the complexed polypeptide or signaling cascade can produce other types of measurable changes in the test composition, such as, for example, activation of a fluorescent signal, changes in pH, measurable release of a reaction product or measurable utilization of a reagent initially present in the aqueous fluid. Of course, there are numerous other modifications that can be measured in various alternative embodiments of the application, depending upon the identity and functionality of the polypeptide and/or signaling cascade being tested.

A wide variety of measuring techniques can also be used, depending upon the type of modification being measured. For example, for measuring certain protein modifications, two-dimensional gel electrophoresis can be used to separate protein populations on the basis of charge and molecular weight. As one example of the use of electrophoresis, phosphorylation changes the protein charge and is often indicated by a horizontal trail of protein spots on a two-dimensional gel. In an analogous manner, changes in the separation properties of peptides that occur upon phosphorylation can be assessed by thin layer chromatography or high performance liquid chromatography, and thus be used to follow the progress of the modification. To study the modifications of a single protein, chromatographic purifications, antibody precipitations, or both, can be used to isolate sufficient amounts for testing. Once a protein has been isolated, a variety of techniques can be used to determine the modified amino acids. For example, in some cases, the precise molecular weight of the intact protein can be established by mass spectrometry (“MS”), which measures mass-to-charge ratio (m/z), yielding the molecular weight and the fragmentation pattern of peptides derived from proteins. MS represents a general method for all modifications that change the molecular weight. MS is especially useful if the protein isolated is not too heterogeneous, its mass is less than about 100 kDa, and it is in a buffer that is compatible with MS. As another example of modification measurement, amino-terminal protein sequencing by the classical technique of Edman degradation is useful for determining proteolytic processing. A wide variety of measurement techniques known in the art can be employed in connection with the application, the above examples representing just a few. By extension, any new measurement process that may be introduced to determine the progress of a reaction, a reaction that was measured formerly by methods established in prior art, can reasonably expected to be used in combination with the present application.

The measurement of choice for a given complex, such as, for example, spectrophotometric measurement of ATPase activity, or the incorporation of phosphate groups into test compounds, can be feasibly adapted for use in industry-standard automated plate readers, which can perform absorbance, fluorescence or luminescence readings on a large number of samples in parallel (such as, for example, from about 96 to about 1500 or more samples). It is apparent to those skilled in the art of high-throughput screening methods that template-assembled signaling complexes can be generated by semi-automatically and/or robotically dispensing the reagents, that include the templates, signaling components, and detection reagents, in a sequential fashion. Such an approach will also permit a synchronized initiation of the activity assay, and thus facilitate high-throughput analyses of the conditions that activate and regulate the signaling pathway in the template-assembled signaling system, including, but not limited to, screens for the effects of potential therapeutic agents, and in the manufacture of specialty reagents that are generated through the use of methods and materials described herein.

The template can take a variety of different forms, limited only by the need to provide a suitable platform to which polypeptides can be attached for appropriate interaction in accordance with the application. In one embodiment, the template is a free-standing template. As used herein, the term “free-standing” is used to mean that the template is not supported by an underlying supported material that is different in kind from the material that constitutes the template. In one embodiment, the free-standing template is a suspendable template. The term “suspendable” is intended to refer a template that can be dispersed homogeneously in an aqueous fluid for a period of time sufficient to use the template for its intended purpose as described herein.

Alternatively, optionally and without regard to any other embodiment, one example of a free-standing template contemplated by the application comprises a lipid “vesicle” or “liposome.” The terms “vesicle” and “liposome” are used interchangeably herein to refer to an assembly of lipids, which are a class of molecules either isolated from natural sources or are synthesized, that have the property of organizing into bilayer structures. For purposes of illustration, lipids can be formed into either ‘small’ unilamellar vesicles (SUVs) prepared by sonication, or ‘large’ unilamellar vesicles (LUVs) prepared by extrusion through the restrictions of filter pores. A vesicle can be formed of a single type of lipid, or can include a mixture of two or more different types of lipid molecules that are mixed together before vesicle formation. In one embodiment, a lipid vesicle is used that includes at least two different types of lipids, at least one of which is a nickel-chelating lipid. A vesicle that includes a nickel-chelating lipid is useful as a template in an assembly of histidine-tagged polypeptides onto the outer leaflet of the SUV or LUV membrane bilayer. Assembly of polypeptides onto such a lipid vesicle is also referred to herein as “vesicle binding.” The organization of polypeptides produced by vesicle binding has been found to resemble the environment of the cell membrane inner leaflet sufficiently well to promote the assembly of active signaling complexes and to restore or significantly improve functionality of polypeptides in comparison to that of free polypeptides in a solution or suspension without such a template.

Other examples of suitable free-standing, suspendable substrates include, but are not limited to, other types of lipid assemblies, such as large multilamellar vesicles, self-assembled lipid nanotoubes, supported membranes, and also polymeric materials. Generally, such vesicles or other template architectures can comprise any compound or composition providing amphiphilic properties, capable of bilayer membrane formation, modified as described herein or as would otherwise be known in the art for specific binding affinity with a suitably-modified receptor component.

Another example of a free-standing template contemplated by the application is a polymer vesicle. The term “polymer vesicle” as used herein refers to a vesicle that has the same topological organization as one formed with lipids, but the membrane layer between the interior and exterior is composed of a synthetic polymer. Examples of polymers, which can be used to form vesicles include, but are not limited to polyethylene oxide-polyethylene diblock copolymers, polyethylene oxide-polybutadiene diblock copolymers, and polyethylene oxide-polypropylene oxide-polyethylene oxide triblock copolymers.

Yet another example of a free-standing template contemplated by the application is a polymer micelle. The term “polymer micelle” as used herein refers to a supramolecular noncovalent assembly that has the recognized organization of a micelle, as for example in a detergent micelle, with a hydrophobic interior and a hydrophilic exterior, and is instead composed of polymer molecules. Examples of polymers, which can be used to form micelles include, but are not limited to polyethylene oxide-polyethylene diblock copolymers and polyethylene oxide-polybutadiene diblock copolymers.

Still another example of a free-standing template contemplated by the application is a polymer molecule. The term “polymer molecule” as used herein refers to a molecule including repeating units joined together by covalent bonds. The polymer molecules that can provide the function of a template have in addition specific sites of attachment for proteins or polypeptides. Such polymers can be synthesized by a variety of methods, such as for example ring-opening metathesis polymerization, or can result from the derivitization of common, available polymers like dextran, branched polyethylene glycol and poly-1-lysine.

Still yet another example of a free-standing template contemplated by the application is a polymer bead. The term “polymer bead” as used herein refers to bead materials that are formed from chemically cross-linked polymers or otherwise self-associated polymers including, but not limited to, polystyrene, polyacrylamide, dextran and agarose.

Alternatively, a template can be formed on a solid substrate, or support (a heterogeneous format). Solid supports include, but are not limited to, supported lipid monolayer and bilayer membranes, self-assembled monolayers (SAMs) and the like. Such supported lipid membranes can be prepared by known methods, including, for example, deposition of monolayer and bilayer membranes on prepared substrates by Langmuir-Blodgett techniques, or through the fusion of vesicles to hydrophobic surfaces in the wells of immunoassay plates. In one example of a template supported on a solid substrate, the substrate is a glass slide. In another example of a template supported on a solid substrate, the substrate is a glass bead. Glass slides and glass beads suitable for use as described herein are readily available commercially. The glass used to form the glass slide or glass bead is preferably borosilicate glass. In another example of a template supported on a solid substrate, the substrate is a silicon wafer. The term “silicon wafer” as used herein refers to disc of silicon that is used in the electronics industry as the substrate for the manufacture of computer chips. In another example of a template supported on a solid substrate, the substrate is a silicon chip. The term “silicon chip” as used herein refers to a portion of a silicon wafer.

In another example of a template supported on a solid substrate, the substrate is a planar noble metal. In another example of a template supported on a solid substrate, the substrate is a colloidal noble metal. Planar noble metal substrates and colloidal noble metal substrates are also available commercially. In another example of a template supported on a solid substrate, the substrate is an oxide layer. The term “oxide layer” as used herein refers to for example the native oxide layer that grows on a surface of a silicon wafers (SiO₂), or various other metal oxides that can be fashioned as layers on surfaces, such as Indium Tin Oxide, or in the form of particles, such as Titanium oxide or Iron oxide. In another example of a template supported on a solid substrate, the substrate is a nanoparticle. The term “nanoparticle” as used herein refers to a colloidal particle that has dimensions no greater than 1000 nm across the largest dimension and more typically no larger than 100 nm across its largest dimension. In another example of a template supported on a solid substrate, the substrate is a polymer slab. In another example of a template supported on a solid substrate, the substrate is a polymer bead. Polymer films and polymer beads suitable for use as described herein are readily available commercially. The polymer used to form the polymer film or polymer bead is preferably polyacrylamide or polyethylene glycol or branched polyethylene glycol.

The polypeptide selected for use is one that corresponds to a membrane-associated protein of interest. The polypeptide can be an entire protein or a fragment of a protein. For example when the membrane-associated protein of interest is a transmembrane protein, the polypeptide selected for attachment to a template is preferably a fragment composed of residues that do not span the cell membrane, i.e. a cytoplasmic (i.e., intracellular) fragment (CF), an extracellular fragment (EF), or more generally an extramembranous fragment. In the case of systems directed to cell transmembrane receptor proteins, for example, the cytoplasmic fragment will often be the functional unit of the protein that is of interest. Furthermore, the polypeptide can be one that provides enzyme (catalytic) activity, that functions as a substrate, or that has recognition motifs for recruitment of other proteins or reagents, such as, for example, signaling and/or adaptor proteins.

With regard to receptor proteins, receptors are instrumental in recruiting cytoplasmic signaling elements, adaptor proteins, enzymes and membrane-associated proteins, into arrangements that modulate pathway activity. Accordingly, the template-assembly methods described herein are applicable to the study of these and other such signaling pathways. For example, Type I receptor proteins are organized with one or more structural domains, which are found on the both sides of the membrane and are joined by a transmembrane segment. An intracellular domain may often have enzymatic activity including tyrosine kinase activity, serine/threonine kinase activity, phosphotyrosine phosphatase, or phosphoserine/threonine phosphatase activity. The tyrosine kinase catalyzes the transfer of the y-phosphate group of adenosine triphosphate (ATP) to tyrosine moieties found within the receptor and to tyrosine containing substrates that dock onto the receptor. Various receptor systems have one or more of the properties of (i) recruitment, (ii) catalytic activity, and (iii) activity as a substrate. Generally speaking the receptors can have all the combinations of properties (i), (ii) and (iii).

Various classes of receptors that are subject to studies described herein include, without limitation, the following: Cytokine Type 1 receptors, Cytokine Type 2 receptors, GPI-anchored, Guanylyl Cyclase receptors, Interleukin-17 receptors, Integrins, Low-density lipoprotein (LDL) receptor and LDL receptor-related proteins, LINGO coreceptors for Nogo/p75, LRR-Ig Receptors, Netrin receptors, Neurexins, Notch, Patched, Plexins, Roundabout, Receptor-like protein tyrosine phosphatases (RPTPs), Receptor Tyrosine Kinases (RTK), Seven transmembrane (7™) receptors, TGF-beta serine/threonine kinase receptors, Tetraspanins, TNF/NGF, and Toll.

In one aspect of the application, the membrane-associated protein under review is a multi-pass transmembrane protein. For example, with reference to the above list of receptors, receptor families 7™ and Patched include multi-pass transmembrane proteins. As used herein, the term “multi-pass” refers to a protein having more than one transmembrane domain, and thus includes one or more loops in the interior side or exterior side of the membrane. For example, 7™ receptors have seven discreet and highly predictable transmembrane domains. In one aspect of the application, the activity of a multi-pass transmembrane protein is analyzed by forming a complex in which the polypeptide corresponds to a loop of the protein, and is attached to the template at one or both ends. In another embodiment, multiple loops and/or fragments of a multi-pass protein are attached to the same template to allow for the loops and/or fragments to complex with one another, or to otherwise function together, to provide a desired activity for analysis.

Another class of receptors to which the present application is well suited is the class of proteins identified as Receptor Tyrosine Kinases (RTK). This large and diverse family of receptors exemplifies several general principles of receptor-ligand and receptor-receptor interactions. All members of the large receptor tyrosine kinase (RTK) family have a similar cytoplasmic catalytic domain that is activated by conformational changes upon ligand engagement whereas members within each subfamily have homologous extracellular domains. No RTKs are found in yeasts or plants. In mammals, multicellular organization is highly dependent on the proper functioning of RTKs as many RTKs have been reported to become oncogenic when their activity is altered. A tyrosine kinase is an enzyme that can transfer a phosphate group from ATP to a tyrosine residue in a protein. These enzymes are a subgroup of the larger class of protein kinases. Phosphorylation is an important function in signal transduction to regulate enzyme activity. The hormones that act on tyrosine kinase receptors are generally growth hormones and factors that promote cell division (i.e., insulin, insulin-like growth factor 1, epidermal-derived growth factor). These enzymes are involved in cellular signaling pathways and regulate key cell functions such as proliferation, differentiation, anti-apoptotic signaling and neurite outgrowth. Unregulated activation of these enzymes, through mechanisms such as point mutations or over-expression, can lead to various forms of cancer as well as benign proliferative conditions. The importance of RTKs in health and disease is further underscored by the existence of aberrations in PTK signaling occurring in inflammatory diseases and diabetes.

RTKs possess an extracellular ligand binding domain, a transmembrane domain and an intracellular catalytic domain. The transmembrane domain anchors the receptor in the plasma membrane, while the extracellular domains bind growth factors. Characteristically, the extracellular domains are comprised of one or more identifiable structural motifs, including cysteine-rich regions, fibronectin III-like domains, immunoglobulin-like domains, EGF-like domains, cadherin-like domains, kringle-like domains, Factor VIII-like domains, glycine-rich regions, leucine-rich regions, acidic regions and discoidin-like domains. The intracellular kinase domains of RTKs can be divided into two classes: those containing a stretch of amino acids separating the kinase domain and those in which the kinase domain is continuous. Activation of the kinase is achieved by ligand binding to the extracellular domain, which induces dimerization of the receptors. Receptors thus activated are able to autophosphorylate tyrosine residues outside the catalytic domain via cross-phosphorylation. The results of this autophosphorylation are stabilization of the active receptor conformation and the creation of phosphotyrosine docking sites for proteins which transduce signals within the cell. Signaling proteins which bind to the intracellular domain of receptor tyrosine kinases in a phosphotyrosine-dependent manner include RasGAP, P13-kinase, phospholipase C, phosphotyrosine phosphatase SHP and adaptor proteins such as Shc, Grb2 and Crk.

One type of RTK to which the present application has been advantageously applied is an Ephrin type-B receptor 2 precursor. Ephrin receptors and their ligands, the ephrins, mediate numerous developmental processes, particularly in the nervous system. Based on their structures and sequence relationships, ephrins are divided into the ephrin-A (EFNA) class, which are anchored to the membrane by a glycosylphosphatidylinositol linkage, and the ephrin-B (EFNB) class, which are transmembrane proteins. The Eph family of receptors are divided into 2 groups based on the similarity of their extracellular domain sequences and their affinities for binding ephrin-A and ephrin-B ligands. Ephrin receptors make up the largest subgroup of the receptor tyrosine kinase (RTK) family. The protein encoded by this gene is a receptor for ephrin-B family members.

Another type of RTK to which the present application has been advantageously applied is an Insulin receptor precursor. After removal of the precursor signal peptide, the insulin receptor precursor is post-translationally cleaved into two chains (alpha and beta) that are covalently linked. Binding of insulin to the insulin receptor (INSR) stimulates glucose uptake.

In Table 1 below, receptors are classified according to taxonom, function and family. The list is not exhaustive, yet it serves to indicate the many examples of biological signal transduction that are conveyed through membrane-associated assemblies. The unifying principles of membrane protein organization and function, generates a reasonable expectation that the present application can be applied to these systems.

TABLE 1

| | | | | |
|---|---|---|---|---|
| | | | Representative | |
| Taxonom | Function | Family | Ligands | Features |

Prokaryotic

| | | | |
|---|---|---|---|
| Recruiters | Methyl-accepting | Aspartic acid, Ni, | Ligands include, light, |
| | Chemotaxis proteins | Co, serine, ribose, | various small molecules, |
| | | galactose, arginine, | pH, internally sensed |
| | | periplasmic | redox potential. MCP- |
| | | binding proteins | based systems mediate |
| | | | chemotaxis, social |
| | | | behavior, gene regulation |
| Enzymes | Two-component sensor | Osmolarity, nitrite, | Primarily control |
| | proteins | nitrate, phosphate | transcription |

Eukaryotic

| | | | |
|---|---|---|---|
| Recruiter with | 7TM | Gonadotropin, | Ligands from light to large |
| 2^{nd} messengers | | gonadotropin- | proteins |
| | | releasing hormone, | |
| | | bradykinin, | |
| | | chemokines, | |
| | | dopamine, | |
| | | adrenergic | |
| | | reagents, light | |

Metazoan Specific

| | | | |
|---|---|---|---|
| Enzymes | Receptor Tyrosine Kinase | EGF, ephrins, FGF, | From close cell-cell |
| | | PDGF, VEGF, | interactions to endocrine |
| | | neuregulins, | signaling |
| | | insulin, IGF-1 | |
| Enzymes | Ser-Thr kinase | TGF-b, BMPs, | Two to three co-receptors |
| | | GDFs | |
| Enzymes | Guanyl cyclase | Natriuretic | cGMP as 2^{nd} messenger |
| | | peptides | |
| Enzymes | RPTP | Midkine, | Most receptors are |
| | | pleiotrophin | orphans |
| Recruiters | Toll | Bacterial | Innate immune |
| | | lipopolysacharide | recognition with NFκB |
| | | | activation |
| Recruiters | LDL/LRP | LDL | Multiligand receptors that |
| | | | can serve as nutrient or |
| | | | signaling co-receptors |
| Recruiters | Integrins | Extracellular | Clustering upon ligand |
| | | matrix | activation |
| Recruiters | Roundabout | Slit | Repellant Interaction |
| Recruiters | Plexins | Semaphorins | Receptors and ligands |
| | | | with sequence similarity |
| Nuclear | Notch | Delta | “One-time” receptor |
| translocation | | | |
| Transporter-like | Patched | Hedgehof- | 12TM protein with a 7TM |
| | | cholesterol | protein smoothened as |
| | | complex | partner |

Chordate specific

| | | | |
|---|---|---|---|
| Recruiters | Cytokine type I | CH, prolactin | Four sets of subunits: α, β, |
| | | (PRL), granulocyte | γ, gp 130: varying |
| | | macrophage- | combinations |
| | | colony stimulating | |
| | | factor (GM-CSF), | |
| | | IL-2 | |
| Recruiters | TNF | TNF | Contain death domain |

Vertebrate specific

| | | | |
|---|---|---|---|
| Recruiters | T cell receptor | Foreign antigens | Cooperate with presenting |
| | | | cells, CD4-CD8 |
| Recruiters | Cytokine type 2 | IFNs, IL-10, IL-22 | Receptor heterodimers |
| | | | bind homodimeric ligands |

As stated above, a complex is provided by attaching one or more polypeptides to a template. In order to do so, it is typically necessary to first modify the polypeptide to include a linker moiety. As used herein, the term “linker moiety” is used to refer to a component effective to interact with the template to attach the polypeptide thereto. A wide variety of linker components are contemplated and a few examples thereof are described below, but these examples are in no way meant to limit the scope of the concept by which proteins and templates can be brought together as described herein.

Examples of ways the polypeptide can be modified to generate interaction between the polypeptide and the template, thereby engendering template-directed assembly, include the following, without limitation: the polypeptide can be designed to accept naturally occurring anchoring modifications; the polypeptide can be designed to engage in metal chelation, for example, by genetic engineering (histidine tag) or through the introduction of a synthetic chemical moiety that engages in chelation; the polypeptide can be engineered for specific covalent attachment to the template; the polypeptide can be engineered to attach a moiety that then engages in a specific covalent to the template; or the polypeptide can be engineered to include or be linked to an insertion domain.

In one embodiment of the application, the linker component is effective to covalently bond to the template, as depicted schematically in FIGS. 3B, C & D. The linker component can be, for example, a genetically engineered segment including at least one amino acid that either permits the covalent attachment of the receptor fragment to the template, or the covalent attachment of a moiety that engenders specific attachment to the template. An example of the former includes the introduction of a cysteine (Cys) residue, which is known to exhibit specific reactivity toward maleimide, and reacts to form a covalent adduct. The maleimide moiety can be made available as the head group in a synthetic lipid molecule, and thereby facilitate direct covalent attachment of the receptor (via cysteine) to the template. An example of a second mode of attachment is illustrated by the introduction of a known biotinylation recognition sequence (e.g., MSGLNDIFEAQKIEWHE) into a fusion protein, which is subsequently acted upon by E. coli biotin ligase (BirA) in the presence of biotin and ATP to covalently attach biotin into such a genetically-engineered receptor fragment. A biotin-modified receptor fragment may then be attached to the template via streptavidin, which binds with high affinity to both biotin groups in the template and to the biotin group receptor fragment.

In another embodiment, the linker component is effective to interact with the template noncovalently by metal chelation, as depicted schematically in FIG. 3E. In one embodiment, the metal or metal ion is associated with the template. The metal or metal ion can alternatively be associated with the linker component. In one preferred embodiment, the linker component comprises a genetically engineered histidine tag fused to the polypeptide. Templating is promoted through a specific noncovalent interaction with the modified phospholipid DOGS-NTA-Ni²⁺. A histidine-tagged polypeptide randomly distributed in solution will orient on binding to a vesicle outer surface via the Ni-NTA-histidine interaction. Alternatively, fusion proteins may involve naturally-occurring binding domains that are effective to bind to certain lipid molecules, which by analogy to DOGS-NTA-N²⁺, can be incorporated into the template. As another alternative, short peptides of known sequence can be incorporated into the template in a similar manner.

Complexes of this application can, in certain embodiments, comprise a membrane including a phospholipid component comprising a metal moiety selective for an amino acid residue of the fragment component. In certain embodiments, and as used to illustrate the broader aspects of this application, a nickel nitrilotriacetic acid moiety can be used to modify a phospholipid such as but not limited to 1,2-dioleoyl-sn-glycero-3-phosphocholine.

In another embodiment, the linker component is effective to interact with the template noncovalently by other complementary interactions. In another embodiment, the linker component is an insertion domain effective to interact with the template noncovalently by insertion of at least a portion of the domain into the template, as depicted schematically in FIG. 3F. The insertion domain can be of a type, for example, wherein at least a portion of the insertion domain interacts with the template by hydrophobic interactions. The insertion domain can be, for example, a genetically engineered peptidyl insertion domain. In another embodiment, the insertion domain comprises an anchoring moiety formed by the adaptation of a naturally occurring mechanism such as, for example, palmitoylation, myristoylation, prenylation, geranylation and GPI linkage.

Palmitoylation is the covalent attachment of fatty acids to cysteine residues of membrane proteins. Palmitoylation increases the hydrophobicity of proteins and contributes to their membrane association. It is a protein modification that is believed to be involved in the control of protein trafficking, localization, partitioning into domains, protein-protein interactions and functions. Palmitoylation of transmembrane proteins typically occurs on cysteine residues located in the border region between the transmembrane region (TMR) and the cytoplasmic domain. Palmitoylation of some proteins is reversible with cycles of acylation and deacylation. Some proteins are palmitoylated in vitro with Pal-CoA in the absence of any enzyme source (Dietrich and Ungermann, 2004). For example, a 100-fold enriched enzyme preparation (PAT) and the photoreceptor rhodopsin can reportedly be used as substrate to compare enzymatic and autocatalytic palmitoylation in vitro. Rhodopsin is palmitoylated with Pal-CoA alone, but addition of the enzyme preparation has been reported to increase the efficiency of acylation approximately 10-fold.

Myristoylation is an irreversible, post-translational protein modification found in animals, plants, fungi and viruses. In this protein modification, a myristoyl group (derived from myristic acid) is covalently attached via an amide bond to the alpha-amino group of an N-terminal glycine residue of a nascent polypeptide. The modification is catalyzed by the enzyme N-myristoyltransferase, and occurs most commonly on glycine residues exposed during co-translational N-terminal methionine removal. Myristoylation also occurs post-translationally, for example when previously internal glycine residues become exposed by caspase cleavage during apoptosis. Myristoylation plays a vital role in membrane targeting and signal transduction in plant responses to environmental stress.

Myristoylation is a very important lipid modification at the N-terminus of eukaryotic and viral proteins. It is involved in directing and anchoring proteins to membranes and, as a consequence, cellular regulation, signal transduction, translocation, several viral induced pathological processes and even apoptosis. The enzyme myristoylCoA:protein N-myristoyltransferase (NMT) recognizes certain characteristics within the N-termini of substrate proteins and finally attaches the lipid moiety to a required N-terminal glycine.

Prenylation or isoprenylation is the addition of hydrophobic molecules to a protein to facilitate its attachment to the cell membrane. The result is similar to that of all lipid anchored proteins (e.g. the GPI anchor). All isoprenylation chains are products of the HMG-CoA reductase pathway: geranylgeraniol (GG), famesol and dolichol.

A GPI anchor or glycosylphosphatidylinositol is a common posttranslational modification of the C-terminus of membrane-attached proteins. It is composed of a hydrophobic phosphatidyl inositol group linked through a carbohydrate containing linker (glucosamine and mannose linked to phosphoryl ethanolamine residue) to the C-terminal amino acid of a mature protein. The two fatty acids within the hydrophobic phosphatidyl-inositol group anchor the protein to the membrane.

During natural processing, glypiated proteins contain a signal peptide, thus directing them into the endoplasmic reticulum (ER). The C-terminus is composed of hydrophobic amino acids which stay inserted in the ER membrane. The hydrophobic end is then cleaved off and replaced by the GPI-anchor. As the protein processes through the secretory route, it is transferred via vesicles to the Golgi and finally to the extracellular space where it remains attached to the exterior leaflet of the cell membrane. Since the glypiation is the sole means of attachment of such proteins to the membrane, cleavage of the group by phospholipases will result in controlled release of the protein from the membrane. The latter mechanism is used in vitro, i.e. the membrane proteins released from the membranes in the enzymatic assay are glypiated protein.

Phospholipase C is an enzyme that is known to cleave the phospho-glycerol bond found in GPI-anchored proteins. Treatment with PLC will cause release of GPI-linked proteins from the outer cell membrane. The T-cell marker Thy-1, acetylcholinesterase, as well as both intestinal and placental alkaline phosphatase are known to be GPI-linked and are released by treatment with PLC. GPI-linked proteins are thought to be preferentially located in lipid rafts, suggesting a high level of organization within microdomains plasma membrane.

As described above, the present application involves the use of a homo- or heterogeneous template to assemble polypeptides, such as, for example, polypeptides derived from membrane-associated proteins, and optionally additional reagents, to provide a functional complex exhibiting the biochemical activity of a membrane-associated protein or protein system, such as, for example, a signaling pathway. The complex can be used to analyze the effect of an active agent on the functionality of the protein or protein system, to analyze the effect of a mutation on the protein or protein system, or to produce reaction products of value in a novel manufacturing process. One embodiment of such a method comprises (1) providing a template configured for attachment of a polypeptide in an aqueous medium; (2) introducing a polypeptide to the medium, the polypeptide having a linker component attached thereto; (3) complexing the polypeptide with the template, and optionally one or more additional components, such as, for example a signaling protein, an adaptor protein or a receptor domain with enzymatic activity; and (4) introducing a test compound into the fluid. In one embodiment, the template is provided by providing a phospholipid component in a medium suitable for vesicle formation, such a component comprising a cationic metal moiety selective for chelation of an amino acid residue, and the polypeptide includes at least one amino acid with affinity for selective coupling bonding or chelating interaction with the phospholipid component. In another embodiment, the polypeptide is covalently bound to the template. In yet another embodiment, the linker component is an insertion domain that interacts with the template to attach the polypeptide to the template.

As stated above, the complex can optionally include additional reagents such as, for example, one or more of a signaling protein, an adapter protein, and/or other membrane-associated components, including other naturally-occurring or synthetic lipids. As used herein, the term “signaling protein” refers to a protein that is part of a cellular signal transduction pathway. In certain embodiments, the signaling protein can be a kinase of the growth factor, or cytokine signaling pathway. The term “adaptor protein” is used to refer to a protein, which is part of a signal transduction pathway, which helps to recruit other proteins in the pathway to the membrane. The signaling protein is typically an enzyme active in or having a role in a particular cellular signal transduction pathway. Where conducive to biochemical activity, such a complex can comprise a mixture of receptor fragments and/or other membrane-associated components, including other naturally occurring lipids and adaptor proteins.

Templates are expected, and shown in the Examples described below, to be compatible with reagents currently used in the assay of enzyme activity. By reference to the Table 2, below, the templates were combined with these other reagents in a solution, and under such conditions generated significant improvements in biochemical functionality. More generally, liposomes, polymerosomes and other templates are expected to be robust and perform in the aqueous solutions in which the biochemical test reagents and proteins are dissolved and assayed (Discher and Eisenberg, 2002; Duzgunes, 2003, 2004).

TABLE 2
Table of Conditions used to Assay Receptor Tyrosine Kinases

| Insulin Receptor | EphB2 | Tie2 |
|---|---|---|
| 55 mM Tris-HCl | 2 mM Tris-HCl | 4 mM Tris-HCl |
| 15 mM NaCl | 6 mM NaCl | 12 mM NaCl |
| 10 μM EGTA | 4 μM EGTA | 8 μM EGTA |
| 0.003% Brij-35 | 0.0018% Brij-35 | 0.003% Brij-35 |
| 27 mM Sucrose | 10.8 mM Sucrose | 21.6 mM Sucrose |
| 20 μM PMSF | 8 μM PMSF | 16 μM PMSF |
| 100 μM Benzamidine | 40 μM Benzamidine | 80 μM Benzamidine |
| 0.01% BME | 0.004% BME | 0.008% BME |
| 100 mM Sodium Orthovanadate | 8.2 mM MOPS | 8.4 mM MOPS |
| 8.4 mM MOPS | 0.1 mg/mL polyGlu₄Tyr | 0.1 mg/mL polyGlu₄Tyr |
| 25 μM Axltide | 10 mM MnCl₂ | 0, 0.125, 0.25, or 0.5 mM MnCl₂ |
| 0.1% glycerol | 0.3% glycerol | 0.01% glycerol |
| 270 nM Insulin Receptor RTK | 77 nm EphB2 RTK domain | 136 nM Tie2 RTK domain |
| domain | 60 μg/mL BSA | 20 μg/mL BSA |
| 20 μg/mL BSA | 60 μM TCEP | 20 μM TCEP |
| 20 μM TCEP | 90 μM ATP | 90 μM ATP |
| 90 μM ATP | 13.5 mM MgCl₂ | 13.5 mM MgCl₂ |
| 13.5 mM MgCl₂ | | |

| TEMPLATES | TEMPLATES | TEMPLATES |
|---|---|---|
| Vesicles: | Vesicles: | Vesicles: |
| 1.7 μM DOGS-NTA-Ni²⁺ | 1.2 μM DOGS-NTA-Ni²⁺ | 1.9 μM DOGS-NTA-Ni²⁺ |
| 1.7 μM DOPC | 1.2 μM DOPC | 1.9 μM DOPC |

Assays using methods and materials as described herein are conducted in a manner similar to that which is known to those skilled in the art of biochemical signaling pathways, and typically follows standard practices, except for the introduction of template reagent in amounts appropriate for the assay.

Reference will now be made to the following Examples, which describe laboratory work that has been performed in support of this application. It is understood that no limitation to the scope of the application is intended thereby. The Examples are provided solely to promote a full understanding of the concepts embodied in the application.

### EXAMPLES

In the illustrative examples 1-3, described below, the concepts described herein are applied to five protein reagents, which comprised purified or partly purified recombinant proteins. Each comprised a different receptor tyrosine kinase (RTK) domain from the Insulin Receptor, the Axl Receptor, the EphB2 Receptor, the ErbB4 receptor, or the Tie2 receptor. All five RTK domains were purchased from Upstate Cell Signaling Solutions Inc. (catalog #: Insulin Receptor, 14-466; Axl Receptor, 14-512; EphB2, 14-553; ErbB4, 14-569; Tie2, 14-540) and had been engineered, for the purpose to aid in purification a hexahistidine tag at the N-terminus of the RTK domain.

In the aforementioned examples described herein, and at least in part as applies to one or more other examples, the hexahistidine tag was used to facilitate an interaction with a template. In the examples described here, templates were lipid vesicles prepared by the method of extrusion, and were comprised of a 1:1 molar ratio of DOPC and DOGS-NTA-Ni²⁺. DOGS-NTA-Ni²⁺ provided the specific affinity for the hexahistidine tag at the N-terminus of the RTK domain, and generated the novel templating interaction that resulted in the improved functionality afforded by the present application. Further background information relating to the experimental work described below is provided in U.S. Pat. No. 7,678,540, which is hereby incorporated by reference herein in its entirety.

Receptor tyrosine kinase domains were tested for autophosphorylating and substrate phosphorylating activities in solution, and in the presence of vesicle templates, under the reaction conditions described in the table above. The procedures were patterned after those used for assaying the reagents in solution, with the exception of the addition of template. No additional incubation periods were required. In all situations the progress of the reaction was measured by phosphate group incorporation, which resulted from the transfer of ³²P-labeled gamma phosphate groups from ATP to either tyrosine moieties within the RTK domain itself, or to tyrosine moieties of substrate reagents, which were, in these examples, either poly([Glu₄Tyr)ₙ or Axltide. The amount of ³²P-phosphate incorporated in all these examples was measured by calibrated scintillation as acid-precipitatable phosphate trapped on disks of filter paper, according to standard procedures known to those skilled in the art.

### Example 1

### Insulin Receptor Autophosphorylation and the Preparation of Phosphorylated Insulin Receptor RTK Domain

FIG. 5A shows the differences in the extent of phosphate group incorporation, after a ten minutes, for reactions conducted the absence of template (in solution) versus reactions conducted in the presence of template. In addition, the reactions were conducted in the presence of the substrate peptide, Axltide, (columns one and two in FIG. 5A), and also in the absence of the Axltide substrate (columns three and four in FIG. 5A). In the absence of the substrate peptide, the incorporation of phosphate is a result of RTK autophosphorylation, which refers to a reaction in which phosphate groups are transferred to certain tyrosine residues in the RTK domain. In this example, the RTK domain is particularly effective at autophosphorylation in the presence of template (last column) and particularly ineffective at autophosphorylation in the absence of template (third column). In a second instance of this reaction, which is provided in support of efficient Insulin Receptor RTK domain autophosphorylation, the data in FIG. 6 are provided. Here also, the autophosphorylation was ineffective in solution, and showed no incorporation over nonspecific background samples. Thus, the improvement factor for autophosphorylation shown in FIG. 6 (Insulin—AutoP, 18,000%) represents the increase over this background incorporation. The amount of phosphate group incorporation in the presence of template is evidence that the template provides for an efficient means to synthesize phosphorylated Insulin receptor RTK domain. The application thus provides a novel means to manufacture this reagent, which is not possible with current methods.

### Example 2

### Improved Regulation of Tie2 RTK Domain Activity, Engendered by Template

FIG. 5B and FIG. 5C depicts the influence of template on the tyrosine kinase activity of the Tie2 RTK domain. The tyrosine kinase activity of the Tie2 RTK domain was measured in the presence of a phosphate-accepting substrate, in this example poly([Glu₄Tyr)ₙ (FIG. 5B), and in its absence, which measured RTK domain autophosphorylation (FIG. 5C). FIGS. 5B and C represents the amount of acid precipitatable phosphate after a 10 minute reaction, as a function of various concentrations of the reagent MnCl₂. These results show that the effect of template can be influenced by the reaction conditions, and provide evidence that the performance of the system can be improved through systematic variations of these conditions, which are known to those skilled in the art.

In the presence of template, the autophosphorylation of the Tie2 RTK domain (FIG. 5C) increases 9-fold at the 0.5 mM Manganese Chloride concentration. As in Example 1, this example demonstrates that the use of the template provides for a new method to prepare phosphorylated protein reagent: phosphorylated Tie2 RTK domain.

This example also depicts improvements in the biochemical functionality of the Tie2 RTK domain, which is engendered by the introduction of template. FIG. 5B shows the total phosphate incorporation, which was regarded as the amount of acid-precipitatable phosphate in the presence of Tie2 RTK domain and the substrate poly([Glu₄Tyr)ₙ. Template serves to lower total phosphate group incorporation compared to the situation in the absence of template (in solution) (FIG. 5B). A comparison of FIG. 5B to FIG. 5C provides evidence that phosphate group incorporation at 0.5 mM Manganese Chloride, in the presence of substrate and template (FIG. 5B right column of the pair), is equal to the phosphate group incorporation at 0.5 mM Manganese Chloride in the absence of substrate and in the presence of template (FIG. 5C right column of the pair). It can thus be deduced from these results that autophosphorylation is the principal reaction event in the presence of template, whether or not substrate is also present. Consequently, the efficient autophosphorylation of the Tie2 RTK domain (FIG. 5C right columns of each pair) leads to inhibition of RTK domain activity and substrate (Poly([Glu₄Tyr)ₙ) phosphorylation. This observation is consistent with current thinking on the regulation of Tie2 RTK activity by autophosphorylation, which is that the autophosphorylation event results in autoinhibition of kinase activity. We infer that the Tie2 RTK domain is not properly regulated in solution, but that via autophosphorylation, which promoted by the template, becomes properly regulated. The application thus provides a means for improving the biological functionality RTK domains. Receptor tyrosine kinase activity of an RTK domain can either be activated or inhibited by autophosphorylation. In the case of the Tie2 RTK domain, activity is inhibited by autophosphorylation. In the case of the Insulin receptor, activity is increased by autophosphorylation. The application provides a means to improve the biological functionality in both of these, as well as other situations.

### Example 3

### Generality of the Effect of Template on RTK Domain Activity

In this example (FIG. 6), the introduction of template is demonstrated to improve the activity of RTK domains from the EphB2 receptor (FIG. 6 top), the Axl receptor (FIG. 6, second from top), the ErbB4 receptor (FIG. 6, third from top) and the Insulin Receptor (FIG. 6, bottom). In each of these cases the introduction of templates improved the tyrosine kinase activities of the RTK domains in the absence of additional substrate (AutoP), that is to say in the autophosphorylation mode, and also in the presence of an added phosphate accepting substrate, either Axltide (with Axl and the Insulin receptor) or poly([Glu₄Tyr)ₙ (EphB2 and Erb4). Improvements ranged between 20% and 700% in the presence of the substrate, and between 70% and 18,000% in the process of autophosphorylation.

As relates to several of the following examples, the PI3K/PDK1/AKT signaling pathway has an important regulatory role in cancer cell growth and tumorigenesis. Signal transduction through this pathway requires the assembly and activation of PDK1 and AKT at the plasma membrane. Upon activation of the pathway, PDK1 and AKT1/2 translocate to the membrane and bind to phosphaditylinositol-(3,4,5)-trisphosphate (PIP₃) through interaction with their pleckstrin-homology (PH) domains. A biochemical method was developed to measure the kinase activity of PDK1 and AKT1/2, utilizing nickel-chelating coated lipid vesicles as a way to mimic the membrane environment. The presence of these vesicles in the reaction buffer enhanced the specific activity of the His-tagged PDK1 (both full-length protein, and a engineered construct consisting of the kinase domain), and the activity of the full length His-tagged AKT1 and AKT2 when assayed in a cascade-type reaction. This enhanced biochemical assay is also suitable to measure the inhibition of PDK1 by several selective compounds from the Carbonyl-4-Amino-Pyrrolopyrimidine (CAP) series. One of these inhibitors, PF-5168899, was used to analyze the effect of PDK1 inhibition on the activation of Akt in the cascade-type reaction.

The phosphoinositide-3 kinase (PI3K)/PDK/AKT pathway is a critical cellular pathway involved in various cell functions such as cell survival, cell differentiation, cell growth and protein expression. The activation of this pathway starts at the cell membrane and is initiated upon the binding of growth factors to their respective tyrosine kinase receptors (RTK's), such as the epidermal growth factor receptor (EGFR), the insulin-like growth factor receptor-1 (IGFR-1), and the insulin receptor (IR) (FIG. 7). Upon binding, these RTK's activate downstream PI3Kα, which catalyzes the phosphorylation of phosphatidylinositol-(3,4)-biphosphate (PIP₂) to generate biologically active phosphaditylinositol-(3,4,5)-trisphosphate (PIP₃). The formation of PIP₃ triggers membrane-based co-localization of the 3′-phosphoinositide dependent kinase-1 (PDK1) and AKT, which bind to PIP₃ through their pleckstrin homology (PH) domains. PDK1 is constitutively activate in the cell due to its the ability to autophosphorylate its own T-loop; however, the migration of this enzyme to the membrane helps to activate AKT1 in conjunction with the mammalian target of rapamycin (mTOR) complex 2 (TORC2) through the phosphorylation of three key residues, Thr308, Ser473 and Thr450. Conversely, the tumor suppressor PTEN (phosphatase and tensin homolog deleted on chromosome 10) down regulates the entire PI3K/AKT pathway. PTEN dephosphorylates PIP₃ and thus prevents the co-localization of AKT and PDK1. In addition, PDK1 has the ability to be recruited in the nucleus. This mechanism is driven by the phosphorylation of key residues on the enzymes such as Ser396, Tyr9 and Tyr376, and by nuclear export signal (NES) in the PDK1 itself. Lastly, the SHP-1 phosphatase has also been shown to associate with the tyrosine phosphorylated PDK1 to facilitate its entry to this cellular compartment. In the nucleus, PDK1 is suspected to phosphorylate specific substrates and to provide protection to the cells against pro-apoptotic stimuli.

Not surprisingly, the aberrant, constitutive activation of the PI3K/AKT pathway plays a major role in the development and survival of various types of cancers due to either the loss of PTEN activity, or to the increase of PI3K and/or AKT activity. For instance, AKT1 gene amplification and mutation occurs in gastric and colorectal cancer, while AKT2 gene amplification has been observed in breast, ovarian and pancreatic cancers. In addition, mutations in PI3Kα or PTEN genes lead to aberrant proliferative signals and cellular transformation. Currently, several AKT1, mTOR, and PI3Kα inhibitors have been reported in the literature, and a few are now either in pre-clinical or in advanced clinical stages. While no late stage and selective inhibitor has been reported for PDK1, it nevertheless represents an attractive target for drug development. PDK1 belongs to the AGC kinase family and was first identified by Phil Cohen's group in 1997. The AGC group is named after the protein kinase A, G, and C families (PKA, PKC, PKG), which are cytoplasmic serine/threonine kinases. Akt1 and Akt2, also referred to as PKBα and PKBβ respectively, are members of the protein kinase B (PKB) family of serine-threonine kinases. Akt1 is implicated in cell survival, inhibition of apoptosis and the induction of protein synthesis. Akt2 is involved in insulin signaling and the induction of glucose transport. PDK1 has been characterized as a master kinase, due its propensity to activate other important downstream AGC kinases such as AKT, P70 ribosomal S6 kinase (S6K), serum and glucorticoid stimulated protein kinase (SGK), atypical and typical PKC, and p90 ribosomal S6 kinase (RSK). RNA antisense targeted against PDK1 in PTEN null cells significantly reduced their proliferation and survival, while overexpression of PDK1 in epithelial cells results in their transformation. In addition, hypomorphic mutation of PDK1 protected PTEN+/−mice from developing a wide range of tumors. Several non-selective inhibitors for PDK1 have already been reported in the literature and have been shown to block survival of cancer cells.

In the present study, we first used a cell free model system composed of TDA 2.0™ lipid vesicles with nickel-chelating headgroups, that mimics the cellular microenvironment. Controlling the exact composition of the vesicles allowed us to study the mechanism of activation of AKT1 and AKT2 in the presence of PDK1 and mTOR (FIG. 8). Under these conditions, we have been able to study the role that a few key residues play upon the activity and the stability of the AKT enzymes and to observe the influence of PDK1 inhibition upon AKT activation. Also, the potency of several novel inhibitors from the Carbonyl-4-Amino-Pyrrolopyrimidine (CAP) series (FIG. 9) was evaluated against PDK1 (S. Murphy, S. Bailey, S. Bergqvist, B. J. Burke, S. Greasley, N. Kablaoui, J. C. Kath, S. Kazmirski, S. Kupchinsky, M. A. Marx, D. Richter, K. Tran, W. Vernier, G. Alton, S. Baxi, J. Ermolieff, L. Lingardo, J. Xie, M-J. Yin. Novel, Potent and Selective Small Molecule Inhibitors of PDK1. Abstracts of the AACR Annual Meeting; Washington, D.C., USA. (2010) Abstract #753). Comparative studies were conducted with two different assay formats and our data suggest that the presence of lipid particles doesn't affect the potency of these compounds. Overall, the addition of TDA 2.0™ provides an enhanced biochemical assay method for measuring the activity of membrane anchored protein kinases and may be useful for kinase drug discovery and high-throughput screening platforms.

Relating to the inventive methods, complexes and/or assemblies illustrated in Examples 4-6, the corresponding figures and discussion, the following were used.

Reagents and general enzymatic assay conditions—EDTA, Tris and HEPES buffer, dimethyl sulfoxide (DMSO), ATP, DTT, magnesium chloride, and Brij35, were all purchased from Sigma-Aldrich (St Louis, Mo.). Fluorescent labeled AKT substrate (5FAM-GRPRTSSFAEG-CONH2) and PDK1 substrate (5FAM-ARKRERTYSFGHHA-COOH) were purchased from Caliper Life Sciences (Hopkinton, Mass.). The PDK1 Omnia peptide (Ac-Sox-PKTFCGTPEYLAPEVRREPRILSEEEQEMFRDFDYIAD-NH2.) was purchased from Invitrogen Life Technologies (Carlsbad, Calif.). The full length human recombinant inactive (unphosphorylated) N-terminal His-tagged AKT1 (amino acids 1-480) was purchased from Cell Sciences (Canton, Mass.). The full-length human recombinant His-tagged PDK1, the full length human recombinant inactive (unphosphorylated) His-tagged AKT2, active mTOR (amino acids 1360-2549) were purchased from Life Technologies. TDA 2.0™ protein assembly reagent-was purchased from Blue Sky Biotech, Inc. (Worcester, Mass.). Recombinant human His-tagged PDK1 catalytic domain (amino acids 51-359) was made at Pfizer La Jolla.

For the Western blot assay, anti-GST, anti-phospho-AKT Thr308 and Ser473 antibodies were purchased from Cell Signaling (Beverly, Mass.). The anti-phospho-AKT Thr450 antibody is from Abcam (Cambridge, Mass.). The goat anti-Rabbit IgG-AP pAb is from Vector Labs (Burlingam, Calif.), the Goat anti-mouse IgG-AP pAb and the goat anti-rabbit IgG-HRP antibody were from Jackson ImmunoResearch (West Grove, Pa.). The anti-His mouse mAb was from Clontech (Mountain View, Calif.)

Production of polyHis-tagged PDK1 (a.a. 51-359) kinase domain—A nucleotide sequence encoding amino acids 51-359 of human PDK1 was cloned into a custom baculovirus transfer vector that appended the cloned fragment with an N-terminal polyhistidine purification tag (MIYYHHHHHHDYGIPTTENLYFQAL). Recombinant baculovirus was prepared using the Bac-to-Bac method (Invitrogen) and used to infect Sf9 insect cells (at moi=1). Infected cells were harvested after 48 h and stored at −80C. The insect cell pellet Was lysed in 50 mM Tris-HCl, pH 7.4, 200 mM NaCl, 0.25 mM TCEP, containing one ‘EDTA-free’ protease inhibitor tablet (Roche) per 75 mL buffer. The suspension was centrifuged at 5,000 g for 1 h and the target bound to ProBond resin (5 mL; Invitrogen). The resin was washed overnight with 50 mM Tris-HCl, pH 7.4, 400 mM NaCl, 20 mM imidazole-HCl, pH 7.4, 1 mM TCEP, and the bound PDK1 step-eluted by using 50 mM Tris-HCl, pH 7.4, 400 mM NaCl, 250 mM imidazole-HCl, pH 7.4, 1 mM TCEP. PDK1 was concentrated to 2 mL by using an Amicon Ultracel 10K (Millipore) centrifugal concentrator and passed through a BioSep S-3000 gel filtration HPLC column (Phenomenex) equilibrated with 25 mM Tris-HCl, pH 7.4, 250 mM NaCl, 1 mM TCEP. The peak fractions were pooled and the PDK1 concentrated to 2.6 mg/mL. Protein concentration was determined by using the Coomassie Plus Protein Reagent (Pierce) with BSA (2 mg/mL ampoule; Pierce) as standard.

Complex formation and organizational activation of PDK1 enzyme activity by TDA 2.0™ protein assembly reagent—The activity of PDK1 (full length and catalytic domain) was measured with and without TDA 2.0™ in 50 mM Tris buffer, 10 mM MgCl₂, 0.01% Tween-20 pH 7.4 (Tris Buffer) with 5% DMSO and 1 mM ATP. PDK1 (50 nM) with and without TDA 2.0™ (5 μM, initial concentration) was serially diluted 2-fold and added to Tris buffer. 5FAM labeled PDK1 peptide (5FAM-ARKRERTYSFGHHA-COOH, final concentration 5 μM) was added in the reaction media in a 96-well V-bottom plate. The enzymatic reaction was initiated upon addition of ATP (1 mM). An aliquot of the assay mixture was then transferred to a low volume 384-well black plate for determination of the relative amounts of substrate peptide and product phosphopeptide using a Caliper EZ-reader from which the rate of turnover was calculated (micromolar per min). The substrate and product were separated on the basis of charge using upstream and downstream voltages of −2250 V and −500 V, respectively, and a screening pressure of −1.2 psi.

AKT activation in the presence of mTOR and PDK1—Activation of AKT1 and AKT2 were conducted in Tris buffer with 2% DMSO. The reaction was conducted with 25 nM unactivated AKT(1 and 2), 25 nM mTOR, 2.5 nM PDK1, and 2.5 μM TDA 2.0™. The reaction was initiated with 5 μM AKT substrate (SFAM-GRPRTSSFAEG-CONH2) and 1 mM ATP and the rate of substrate conversion was measured on a LabChip EZ-Reader. The instrument was set up to collect aliquots from the assay mixture at regular intervals. The upstream, downstream voltages and the pressure were set to −2800 V, −380 V, and 0.8 psi, respectively.

Determination of the apparent Michaelis-Menten constants, Kₘ^{app} and k_{cat}^{app} for PDK1—Kₘ^{app} and k_{cat}^{app} values for ATP were determined in the presence of 25 nM full-length PDK1 and 2.5 μM TDA 2.0™ in 50 mM Tris buffer, 10 mM MgCl₂, 0.01% Tween-20 pH 7.4 with 5% DMSO. The enzyme was incubated in a 96-well V-bottom plate for 10 min in assay buffer, in the presence of 5 μM 5FAM-PDK1 peptide. The reaction was then initiated by addition of various concentrations of ATP. Product phosphopeptide was determined as previously described (see previous section). Kₘ^{app} and k_{cat}^{app} values for 5FAM labeled peptide were determined using the same experimental condition in the presence of 1 mM ATP and various concentrations of peptide.

Enzyme inhibition—Inhibition studies were conducted using two assay formats, Omnia and Caliper. For the Omnia assay, K₁^{app} studies were conducted in the presence of 20 nM catalytic domain of PDK1, 50 μM ATP and 3 μM PDK1-Onmia-peptide in a 50 mM Hepes, 5 mM MgCl₂, 0.01% Brij-35, 1 mM DTT assay buffer at pH 7.4. The increase of fluorescence (λₑₓ=360 nm and λₑₘ=485 nm) was recorded continuously using a Safire TECAN plate reader. For the Caliper assay, the Kᵢ^{app} constant for full-length PDK1 alone was determined in the presence of 25 nM enzyme. For AKT1, the reaction was conducted with 25 nM inactive AKT1, 25 nM mTOR, 2.5 nM full-length PDK1. Both sets of Caliper inhibition study were conducted with 2.5 μM TDA 2.0™, 1 mM ATP and 5 μM peptide in 50 mM Tris buffer, 10 mM MgCl₂, 0.01% Tween-20 pH 7.4 with 5% DMSO. The enzyme, the peptide and various amounts of inhibitor were preincubated for 15 min, prior addition of ATP to the reaction media.

Kᵢ^{app} and Kᵢ were calculated by fitting the experimental data to the following equations (J. F. Morrison Kinetics of the reversible inhibition of enzyme catalysed reactions by tight-binding inhibitors. Biochim. Biophys. Acta 185, (1969), 269-286):

$$\begin{matrix}Vi = Vo \cdot ( 1 - ( \frac{\begin{matrix}[ E ] o + [ I ] o + {K}_{i}^{app} - \\ \sqrt{{( [ E ] o + [ I ] o + {K}_{i}^{app} )}^{2} - 4 \cdot [ E ] o \cdot [ I ] o}\end{matrix}}{2 \cdot [ E ] o} ) ) & ( 1 ) \\ Where {K}_{i}^{app} = {K}_{i} \cdot ( 1 + [ A o ] / {K}_{m} ) & ( 2 )\end{matrix}$$

[E]ₒ and [I]ₒ are the total active enzyme and inhibitor concentrations, respectively; Kᵢ is the inhibition binding constant; Vᵢ and Vₒ are the rates of peptide phosphorylation in the presence or in the absence of inhibitor, respectively; [A]ₒ is the total ATP concentration.

Western Blots—Assays were conducted for 30 min. at room temperature in 20 mM HEPES pH 7.2, 10 mM MgCl₂, 0.01% Tween-20, 100 μM ATP, and 100 μM Crosstide (Anaspec). Enzyme concentrations for western analysis were as follows 200 nM AKT1 or AKT2, 200 nM mTOR, 20 nM full length-PDK1 and 20 μM TDA 2.0™. Samples from kinase reactions were analyzed by SDS-PAGE (4-20% acrylamide tris-glycine, Lonza) using standard methods. Antibodies used: anti-His (Clontech), Phospho-AKT (Thr308) (C31E5E) (Cell Signaling), Phospho-AKT (Ser473) (D9E) (Cell Signaling), anti-GST (91G1) (Cell Signaling), Goat anti-Rabbit IgG-AP (Vector Labs), Goat anti-Mouse IgG-AP (Jackson Immuno). Immunoreactive bands were visualized using Western Blue® Stabilized Substrate. Western blots were quantitated using ImageJ software.

### Example 4

PDK1 and AKT1/AKT2 activity in the presence of TDA 2.0™—PDK1 activity was measured using a small 14-mer 5FAM labeled peptide (5FAM-ARKRERTYSFGHHA-COOH) in the presence and in the absence of TDA 2.0™. As illustrated in FIGS. 10A and 10B, the addition of lipid based particles in the assay buffer boosts the PDK1 enzyme activity by ˜4-5-fold for the catalytic domain and 20-fold for the full-length enzyme as compared to the enzyme alone. Also, data in FIG. 10C show that the activation occurs only in the presence of His-tagged PDK1. The actual effect of these artificial vesicles on the PDK1 activity remains to be fully understood; however, TDA 2.0™ contain Ni²⁺-chelating moieties creating a template which directs the assembly of purified His-tagged proteins which are normally membrane associated; this approach has been utilized by several research groups with a broad range of protein classes (S. Gridley, A. L. Shrout, and E. A. Esposito, Challenges and approaches for assay development of membrane and membrane-associated proteins in drug discovery. Progress in Molecular Biology and Translational Science, 91, (2010), 209-239). Therefore, it appears that it promotes assembly of relevant membrane-associated conformation, stimulating the trans-autophosphorylation and subsequently the trans-activation of PDK1 via protein co-localization thus replicating the normal cellular effect of PIP₃ recruitment of PDK1 to the membrane via its PH domain. Further kinetic analysis was conducted with full length-PDK1 and TDA 2.0™ to determine a Kₘ^{app} and k_{cat}^{app} values of 13.6±2.7 μM and 0.72±0.024 min⁻¹ for ATP, respectively, and 25.5±5.7 μM and 1.8±0.18 min⁻¹ for the 5FAM-peptide. It was not possible to measure and compare these same constants in the absence of these TDA 2.0™ due to the lack of significant PDK1 activity toward the peptide substrate.

The effect of TDA 2.0™ was also evaluated upon the activation of AKT1 and AKT2 by full length-PDK1 and mTOR. As illustrated in FIGS. 11A and 11B, AKT is readily activated when full length-PDK1, mTOR and TDA 2.0™ are simultaneously present in the reaction media. Interestingly, our experimental data showed that a brief burst of AKT2 activity was also recorded only in the presence of PDK1 and TDA 2.0™ (FIG. 11B); however, the activity of AKT2 plateaued very rapidly, within 20 min., suggesting that enzyme stability is negatively affected when mTOR is absent from the assay buffer. These results are in agreement with previous studies conducted by Facchineti et al (2008) that identify mTOR as a key enzyme responsible for the folding and the stability of AKT (V. Facchinetti et al, The mammalian target of rapamycin complex 2 controls folding and stability of Akt and protein kinase C. EMBO Journal, 27, (2008) 1932-1943).

### Example 5

Western Blot analysis—Western blot analysis phosho-specific antibodies of samples from kinase assays indicates that addition of mTOR and PDK1 with AKT1 increases the level of phospho-Ser473 and phospho-Thr308 (FIG. 12). Addition of TDA 2.0™ significantly increases phosphorylation on these residues as well. Surprisingly, western blot analysis also showed that AKT1 and AKT2 seems to autophosphorylates on Ser473 when TDA 2.0™ is present in the reaction media and that mTOR can phosphorylate both residues, Ser473 and Thr308. Lastly, residue Thr450 on AKT1 and AKT2 appear to be already phosphorylated prior addition of mTOR and PDK1 to the media.

### Example 6

PDK1 and AKT1 inhibition—A few select inhibitors from the CAP series (Table 3, below) were evaluated against full length-PDK1. The mechanism of inhibition of these inhibitors has been resolved by previous crystallography studies which showed these compounds competing with the ATP at the kinase hinge region. Kᵢ for these compounds are reported in Table 3. One of these compounds, PF-5168899, was further evaluated to prevent the activation of AKT1. While, the initial data set showed that the inhibitor can effectively inhibit the PDK1 activity in the nM range at high concentration of ATP (i.e., a Kᵢ^{app} and a Kᵢ values of 31.5±2.4 nM and 0.31±0.04 nM were determined, respectively), the compound is considerably less effective to prevent the activation of AKT1 when used in a cascade assay (i.e. K₁^{app}=1.53±0.66 μM, a ˜70-fold increase as compared to the Kᵢ^{app} for PDK1—see FIG. 13).

TABLE 3
Kᵢ values against PDK1 were determined with select CAP
inhibitors using Omnia and Caliper assay formats. The Kᵢ^{app}
was converted into Kᵢ value using equation 2 for competitive inhibitor.

| PDK1 inhibitors | Kᵢ (nM, Omnia) | Kᵢ (nM, Caliper) |
|---|---|---|
| PF-4350677 | 2.3 ± 1.4 (n = 2) | 10.6 ± 0.5 (n = 2) |
| PF-5168899 | 2.5 ± 1.1 (n = 3)^{a} | 0.3 ± 0.04 (n = 2) |
| PF-5168961 | 0.2 ± 0.02 (n = 1) | 0.6 ± 0.1 (n = 2) |
| PF-5201534 | 1.25 ± 0.55 (n = 4)^{a} | 1.8 ± 0.1 (n = 2) |

^{a}Data from Murphy S et al [20]

As related to examples 4-6, upon activation by RTKs, the recruitment of PDK1 to the membrane triggers a cascade of events that includes the autoactivation of PDK1. In turn, PDK1 phosphorylates and activates several downstream kinases such as AKT, SGK3 and S6K. As described by Wick et al (2003), PDK1 is autoactivated through a series of well-coordinated events that requires the dimerization of the enzyme through the PH domain and trans-autophosphorylation in the activation loop (i.e. A-loop). Several studies have revealed that docking sites such as the PIF domain located on the PDK1 N-terminal domain can also play a critical role in the regulation of the enzyme activity. In particular, the interactions between either large peptides or small ligands with these docking sites induce changes in the protein conformation and lead to an increase of enzyme activity. Interestingly, we have also been able to enhance the enzyme activity by adding TDA 2.0™, in the reaction media. These vesicles were added in order to mimic the cellular environment and to reproduce the cascade of events that leads to the PDK1 activation. As reported in this study, a 4-5-fold & 20-fold increases of enzyme activity was observed in the presence of a small artificial peptide (14-mer) with either the catalytic domain or the full length PDK1, respectively. Although the mechanism of activation of this enzyme remains unclear, it is likely that PDK1 binds to TDA 2.0™ through the His-tag and establishes dimers, or higher order oligomeric structures. The dimerization of this enzyme would be followed by trans-autophosphorylation and autoactivation.

The effect of TDA 2.0™, was also studied using a more intricate biochemical assay that was designed specifically to study the activation of inactive (unphosphorylated) AKT by PDK1 and mTOR kinases. As reported in FIGS. 11A and 11B, the presence of these artificial vesicles significantly boosted the activation of AKT1 and AKT2 activity. Both AKT enzymes showed a burst of activity that quickly plateaued if coupled with PDK1 alone. However, AKT displayed a greater and more linear rate level of activity when both enzymes, PDK1 and mTOR, were both added to the assay (FIG. 11B). Conversely, these two enzymes have very limited impact upon the AKT activation in the absence of these lipids vesicles. To further understand this mechanism of activation, a western blot analysis was conducted in order to identify the phosphorylation state of the key amino acid residues that have been reported to regulate the enzyme activity. The results generated are in agreement with previous studies, which show that PDK1 phosphorylates residue Thr308 in the A-loop of AKT. The phosphorylation of this amino acid residue alone is sufficient to activate AKT to a limited extent, however, the full activation of this enzyme requires the phosphorylation of additional residues such as Ser473 in the C-terminal hydrophobic motif and Thr450 in the turn motif by mTOR and other kinases. As previously reported by Facchinetti et al., 2008, the phosphorylation of residues Thr450 and Ser473 play an important role upon the stability of the enzyme which seems to be consistent with our kinetic and data. Also and similarly to Facchinetti's group, the present study shows that AKT autophosphorylates its own Ser473 residue. Surprisingly, the last piece of data provided by the western blot analysis suggests that mTOR has the ability to phosphorylate both residues Ser473 and Thr308 on AKT (FIGS. 12A & B, columns 7 and 8). The data generated with these liposomes indicate that we have been able to reproduce, to a limited extent and in a chemically-defined in vitro assay, the cascade of events that lead to the in vivo the in vivo activation of AKT. In agreement with recent studies, these data also suggest that the presence of PIP₃ and the PH domain are not needed for activation of PDK1 or AKT. Therefore, we propose that AKT activation is initiated upon binding to TDA 2.0™, which provides a critical membrane context that leads to the exposure of the A-loop and the hydrophobic motif of the C-terminus, conformationally altering AKT to become an optimal substrate for PDK1 and mTOR. The membrane environment afforded by association with TDA 2.0™, and the conformational changes imparted by that association, are likely to be the critical molecular events responsible for activation and pharmacology observed here. Separately, mTOR phosphorylates Ser473 resulting in full activation and increase stability of AKT.

The effect of liposomes on the PDK1 activity was also evaluated in the presence of PDK1 inhibitors from the carbonyl-4-amino-pyrrolopyrimidine series. A comparative study was conducted in two different assay formats, Omnia kinetic assay and Caliper mobility shift assay. The Kᵢ values obtained using the Omnia assay were determined without TDA 2.0™ as opposed to the values determined using the Caliper assay. As reported in Table 3, the values are the same between both assays which demonstrate that while nanoparticles increase the activity of the kinases, the binding and inhibition of that activity by small-molecule inhibitors remained unperturbed. One selective PDK1 inhibitor from the carbonyl-4-amino-pyrrolopyrimidine series, PF-5168899, was also evaluated to prevent the activation of AKT using a cascade biochemical assay. This compound inhibits PDK1 with Kᵢ value in the nM range in the presence and in the absence of lipid vesicles. This inhibitor was used as a tool to evaluate the inhibition of PDK1 upon downstream biomarkers such as the activation of AKT. Surprisingly, our biochemical data show this inhibitor does not seem to affect the activation of AKT to the same extent; this compound is actually 70-fold less potent in preventing the activation of AKT1 in a biochemical cascade assay. The loss of potency from PDK1 to AKT is unclear; however, the western blot data suggests an alternative mode of activation for the AKT enzymes which could be driven by the combination of either PDK1+mTOR or by a mechanism of AKT autophosphorylation and mTOR which was also shown to phosphorylate both residues, Thr308 and Ser473. Under these circumstances, selective inhibition of PDK1 could only have a limited effect upon the rest of AKT pathway.

In conclusion, this study investigates the mechanism of activation of PDK1 and AKT in the presence of TDA 2.0™. We showed that these artificial liposomes enhance the PDK1 activity and could be used in a pseudo in vitro cellular assay to study the activation and/or inhibition of the kinases from the PI3K/AKT pathway. A new class of potent inhibitors of PDK1 was also investigated using two biochemical assay formats and our experimental data showed that addition of the nickel-chelating liposomes is suitable for assaying kinase-signaling pathways in the presence of inhibitors.

These data are additional evidence that vesicles facilitate the assembly of different proteins in close proximity to each other. The close proximity is necessary for function, and does not take place in the absence of templating membranes. The close proximity on the surface of the template can lead to teams of proteins, or complexes, which have stable or transient interactions. The fluid template facilitates either transient or stable interactions among the template-associated proteins, as needed for function.

With respect to the specific mode of activation in this example, the histidine tag—nickel lipid interaction is a surrogate for the cellular mechanism of membrane association, which involves the phosphorylation of PIP₂ by PI3K to generate PIP₃. Both PDK1 and Akt become associated with the membrane through a binding interaction between PIP3 and the PH domains that are found in PDK1 and Akt (FIG. 7). A surrogate membrane-associating system, e.g. the histidine-tag—nickel-lipid interaction, can be used with analogous signaling pathways that generate a membrane association in the cell through specific lipid-protein interactions. Other examples where this may be used include, but are not limited to systems involving the protein kinase C (PKC) family, the TEC family kinases, and the MAP kinases (T. O. Chan, S. E. Rittenhouse, P. N. Tsichlis, AKT/PKB and other D3 phosphoinositide-regulated kinases: kinase activation by phosphoinositide-dependent phosphorylation. Annu. Rev. Biochem. 68, (1999), 965-1014).

Any reference to a specific direction, for example, references to up, upper, down, lower, and the like, is to be understood for illustrative purposes only or to better identify or distinguish various components from one another. Unless specifically identified to the contrary, all terms used herein are used to include their normal and customary terminology. Further, while various embodiments having specific components and structures are described and illustrated herein, it is to be understood that any selected embodiment can include one or more of the specific components and/or structures described for another embodiment where possible.

While multiple embodiments have been described in detail in the foregoing description, the same is to be considered illustrative and not restrictive in character, it being understood that only selected embodiments have been shown and described and that all changes, equivalents, and modifications that come within the scope of the inventions described herein or defined by the following claims are desired to be protected. Any experiments, experimental examples, or experimental results provided herein are intended to be illustrative of the present application and should not be construed to limit or restrict the scope of the claims set forth below. Further, any theory, mechanism of operation, proof, or finding stated herein is meant to further enhance understanding of the concepts described herein and is not intended to limit the present application in any way to such theory, mechanism of operation, proof, or finding. In reading the claims, words such as “a,” “an,” “at least one” and “at least a portion” are not intended to limit the claims to only one item unless specifically stated to the contrary. Further, when the language “at least a portion” and/or “a portion” is used, the claims may include a portion and/or the entire item unless specifically stated to the contrary.

What is claimed is:

1. A method for analyzing in vitro the effect of a molecule upon a polypeptide-catalyzed reaction or cascade, comprising:
providing an aqueous fluid including:
one or more reagent; and
a biologically active complex including a lipid membrane-like template and at least one membrane-associated polypeptide attached to the template, wherein the complex is functional under a given set of conditions to produce a measurable modification in the content of said one or more reagent or in said polypeptide;
introducing into the fluid a test molecule selected from a drug, a drug candidate, an agonist and an antagonist into the fluid; and
measuring the modification to determine the effect of the test molecule on the reaction or cascade,
wherein the test molecule is not a molecule of the biologically-active complex or a variant thereof.

2. The method in accordance with claim 1 wherein the test molecule is selected from a drug and a drug candidate.

3. The method in accordance with claim 1 wherein the measurable modification results from a process selected from (1) a chemical modification to the polypeptide the polypeptide resulting from intrinsic enzymatic activity of the polypeptide as it interacts with the template, (2) chemical modification of a soluble substrate reagent present in the fluid that is catalyzed by the polypeptide as it interacts with the template, (3) chemical modification of a soluble substrate reagent that is catalyzed by enzymatic activity of a signaling enzyme present in the fluid that is recruited to the complex, (4) chemical modification to the polypeptide in a process catalyzed by a signaling protein that is recruited to the complex, and (5) chemical modification of a soluble substrate reagent present in the fluid that resulting from a reaction cascade initiated by the polypeptide as it interacts with the template or a signaling enzyme that is recruited to the complex.

4. The method in accordance with claim 1 wherein the membrane-associated polypeptide is selected from non-receptor tyrosine kinases and serine-threonine kinases.

5. The method in accordance with claim 1 wherein the measurable modification is a modification selected from the group consisting of phosphorylation, dephosphorylation, acetylation, methylation, acylation, glycosylation, glycosylphosphatidylinositol (GPI) anchoring, sulfation, disulfide bond formation, deamidation, ubiquitination, sumoylation nitration of tyrosine, hydrolysis of ATP or GTP activation of a fluorescent signal, release of a reaction product and utilization of a reagent initially present in the fluid.

6. The method in accordance with claim 1 wherein the template is a free-standing template.

7. The method in accordance with claim 6 wherein the template is selected from a phospholipid vesicle, a polymer vesicle, a polymer micelle, and a polymer molecule.

8. The method in accordance with claim 1 wherein the template is supported on a solid substrate material.

9. The method in accordance with claim 8 wherein the substrate is selected from a glass slide, a glass bead, a silicon wafer, a silicon chip, a planar noble metal, a colloidal noble metal, a metal oxide layer, a nanoparticulate material, a polymer slab, a polymer film and a polymer bead.

10. The method in accordance with claim 8 wherein the template is selected from a phospholipid bilayer, a phospholipid monolayer and a polymer film

11. The method in accordance with claim 1 wherein the polypeptide has attached thereto a linker component effective to attach the polypeptide to the template.

12. The method in accordance with claim 11 wherein the linker component is selected from a component effective to covalently bond to the template, a component effective to interact with the template noncovalently by metal chelation, a component effective to interact with the template noncovalently by other complementary interactions, and an insertion domain effective to interact with the template noncovalently by insertion of at least a portion of the domain into the template.

13. The method in accordance with claim 11 wherein the linker component comprises a component effective to interact with the template noncovalently by metal chelation, and wherein the metal or metal ion is associated with the template.

14. The method in accordance with claim 11 wherein the linker component comprises a component effective to interact with the template noncovalently by metal chelation, and wherein the metal or metal ion is associated with the linker component.

15. The method in accordance with claim 11 wherein the linker component comprises a genetically engineered histidine tag.

16. The method in accordance with claim 11 wherein the linker component comprises an insertion domain.

17. The method in accordance with claim 16 wherein the insertion domain is effective to interact with the template noncovalently by insertion of at least a portion of the domain into the template, and wherein at least a portion of the insertion domain interacts with the template by hydrophobic interactions.

18. The method in accordance with claim 16 wherein the insertion domain comprises a genetically engineered peptidyl insertion domain.

19. The method in accordance with claim 16 wherein the insertion domain comprises an anchoring moiety formed by the adaptation of naturally occurring mechanisms.

20. The method in accordance with claim 19 wherein the naturally occurring mechanism is selected from the group consisting of palmitoylation, myristoylation, prenylation, geranylation, GPI linkage and a synthetic analog thereof.

21. The method in accordance with claim 4 wherein the domain is selected from kinases of the P13K/PDK1/Akt pathway.

22. The method in accordance with claim 21 wherein the domain comprises an Akt1 kinase, a fragment thereof, or a functional variant thereof.

23. The method in accordance with claim 21 wherein the domain comprises an Akt2 kinase, a fragment thereof, or a functional variant thereof.

24. The method in accordance with claim 21 wherein the domain comprises an mTOR kinase, a fragment thereof, or a functional variant thereof.

25. The method in accordance with claim 21 wherein the domain comprises a PDK1 kinase, a fragment thereof, or a functional variant thereof.

26. A complex, comprising:
a lipid membrane-like template; and
a polypeptide linked to the template, the polypeptide comprising a human membrane-associated protein or a fragment thereof, or a polypeptide having at least about 80% identity thereto, the polypeptide having attached thereto a linker component that does not substantially affect the functionality of the polypeptide and that is effective to attach the polypeptide to the template.

27. The complex in accordance with claim 26 wherein the polypeptide is derived from a transmembrane receptor protein.

28. The complex in accordance with claim 26 wherein the polypeptide is a cytoplasmic domain derived from a receptor tyrosine kinase.

29. The complex in accordance with claim 28 wherein the polypeptide comprises an insulin receptor protein, a fragment thereof or a functional variant thereof.

30. The complex in accordance with claim 28 wherein the polypeptide comprises an ErbB4 receptor protein, a fragment thereof or a functional variant thereof.

31. The complex in accordance with claim 28 wherein the polypeptide comprises an Axl receptor protein, a fragment thereof or a functional variant thereof.

32. The complex in accordance with claim 28 wherein the polypeptide comprises an EphB2 receptor protein, a fragment thereof or a functional variant thereof.

33. The complex in accordance with claim 26 wherein the polypeptide comprises a domain selected from non-receptor tyrosine kinase domains and serine-threonine kinase domains.

34. A complex, comprising:
a membrane-like template comprising a phospholipid bilayer; and
a polypeptide linked to the template, the polypeptide comprising a human membrane-associated protein selected from non-receptor tyrosine kinases and serine-threonine kinases or a fragment thereof, or a polypeptide having at least about 80% identity thereto, the polypeptide having attached thereto a linker component that does not substantially affect the functionality of the polypeptide and that is effective to attach the polypeptide to the template.

35. The complex in accordance with claim 34 wherein the polypeptide is a kinase of the P13K/PDK1/Akt pathway.

36. The complex in accordance with claim 34 wherein the polypeptide is a cytoplasmic domain derived from a non-receptor serine-threonine kinase.

37. The complex in accordance with claim 36 wherein the polypeptide comprises an Akt1 kinase, a fragment thereof or a functional variant thereof.

38. The complex in accordance with claim 36 wherein the polypeptide comprises an Akt2 kinase, a fragment thereof or a functional variant thereof.

39. The complex in accordance with claim 36 wherein the polypeptide comprises an mTOR kinase, a fragment thereof or a functional variant thereof.

40. The complex in accordance with claim 36 wherein the polypeptide comprises a PDK1 kinase, a fragment thereof or a functional variant thereof.

41. The complex in accordance with claim 34 in an aqueous fluid medium.

42. The complex in accordance with claim 41 wherein said medium comprises a test molecule selected from a drug, a drug candidate, an agonist and an antagonist.
